(19) United States
(12) Patent Application Publication
(10) Pub. No.: US 2026/0021307 A1
(43) Pub. Date: Jan. 22, 2026

(54) IMPLANTABLE PULSE GENERATOR WITH RESIDUAL VOLTAGE MONITORING FOR DIAGNOSTICS FOR PROVIDING A MEDICAL THERAPY TO A PATIENT USING ELECTRICAL PULSES

(71) Applicant: Advanced Neuromodulation Systems, Inc., Plano, TX (US)

(72) Inventors: Daran DeShazo, Lewisville, TX (US); Edward Lundberg, Frisco, TX (US)

(21) Appl. No.: 19/269,889

(22) Filed: Jul. 15, 2025

Related U.S. Application Data
(60) Provisional application No. 63/672,003, filed on Jul. 16, 2024.

(51) Int. Cl. A61N 1/36

(52) U.S. Cl. CPC A61N 1/36139; A61N 1/36142; A61N 1/36062

(57) ABSTRACT

A system and method for residual voltage monitoring and extracting ETI load parametric data relative to one or more electrodes of an implanted stimulation lead system associated with an IPG for diagnostics and for providing a medical therapy to a patient using electrical pulses. Responsive to comparing the measured residual voltages against estimated residual voltages obtained based on the ETI load parametric data, appropriate corrective action may be effectuated.

### CROSS-REFERENCE TO RELATED APPLICATION

This application claims priority to U.S. Provisional Application No. 63/672,003, filed on Jul. 16, 2024, which is incorporated herein by reference in its entirety.

### TECHNICAL FIELD

The present application generally relates to implantable pulse generators for providing a medical therapy to a patient using electrical pulses and circuitry associated therewith.

### BACKGROUND

Implantable medical devices are used for a wide variety of medical conditions. For example, a number of implantable medical devices have been commercially distributed that allow electrical pulses or signals to be applied to neural or other tissue after implantation of the respective device within a patient. Such implantable medical devices may be used for cardiac pace making, cardiac rhythm management, treatments for congestive heart failure, implanted defibrillators, and neurostimulation. Neurostimulation encompasses a wide range of applications, such as for example, treatment of chronic pain, treatment of motor disorders, treatment of incontinence and other sacral nerve related disorders, reduction of epileptic seizures, and treatment of depression.

Neurostimulation in the form of spinal cord stimulation (SCS), for example, has been used as a treatment for chronic pain for a number of years. SCS is often used to alleviate pain after failed back surgery, pain due to neuropathies, or pain due to inadequate blood flow. In spinal cord stimulation (SCS), an implantable pulse generator (IPG) is often employed that typically includes electronic circuitry to generate electrical pulses, a primary cell or rechargeable battery, communication circuitry, one or more processors to control device operations, and other components. Upon implantation for a given patient, the IPG is connected to one or more stimulation leads. The electrodes of the stimulations are positioned within the epidural space of the patient for SCS to apply the electrical pulses generated by the IPG to dorsal column fibers of the patient.

Implantable pulse generators have been the subject of advanced physiological engineering research for the past few decades. Considering the complexity of IPGs combined with the extremely wide range of programming parameters that can be applied by the physician while programming, it is imperative to ensure that the delivered program is safe for the implanted electrode system and patient tissue being stimulated. Since electrode corrosion or damage can occur when too much charge is injected through an electrode, given its effective surface area and material composition, measures must be taken to ensure that unsafe conditions are protected against even in the presence of device faults. This often addressed by conservative hardware mitigations which add unwanted size to the IPG, making it more invasive and less comfortable for the patient.

### SUMMARY

Embodiments of the present patent disclosure are broadly directed to implantable pulse generators or other implantable or minimally/partially implantable medical devices having diagnostic circuitry configured to monitor electrodes' residual voltages (VRES) and utilize the measured VRES data in a variety of diagnostic applications. In some examples, an IPG/IMD may be configured to compare the measured residual voltages against estimated residual voltages obtained based on the electrode/tissue interface (ETI) load parametric data and detect potential fault conditions in a given therapy setting depending on the observed differences between the measured residual voltages and the estimated residual voltages.

In one aspect, an IPG is disclosed for generating electrical pulses for application to tissue of a patient, comprising: a controller for controlling operations of the IPG; a battery for powering the IPG; circuitry for generating electrical pulses; a header for connecting to one or more stimulation leads with a plurality of electrodes; and diagnostic circuitry coupled to the controller and the circuitry for generating electrical pulses. In one arrangement, the diagnostic circuitry may be configured to measure a residual voltage (VRES_{MEAS}) across at least one electrode (e.g., relative to a reference node such as one or more other electrodes depending on the programmed electrodes). In one arrangement, the controller may be configured to estimate a residual voltage (VRES_{EST}) expected to accumulate across the at least one electrode, wherein the estimated VRES_{EST} is determined based on one or more stimulation pulse parameters and one or more impedance parameters associated with an equivalent circuit of an ETI representation of the at least one electrode, and responsive to determining that a difference between the measured VRES_{MEAS} and the estimated VRES_{EST} is greater than a threshold, indicate a potential fault condition with respect to the IPG's operation. Depending on implementation, the potential fault condition may be indicative of a DC leakage fault associated with the at least one electrode, the IPG, or both. In an example arrangement, the IPG may be configured such that each electrode of the plurality of electrodes is coupled to a respective conductive trace disposed in the stimulation lead without having a DC blocking capacitor (C_{DC}) coupled thereto.

In another aspect, a method of operating an IPG is disclosed, wherein the IPG includes one or more leads implanted proximate to a patient's tissue, each lead including a respective set of electrodes. The method may comprise, inter alia, applying one or more stimulation pulses to at least one electrode of the lead system; measuring a residual voltage (VRES_{MEAS}) across the at least one electrode relative to a reference node; estimating a residual voltage (VRES_{EST}) expected to accumulate across the at least one electrode, wherein the estimated VRES_{EST} is determined based on one or more stimulation pulse parameters and one or more impedance parameters associated with an equivalent circuit of an ETI representation of the at least one electrode; and responsive to determining that a difference between the measured VRES_{MEAS} and the estimated VRES_{EST} is greater than a threshold, indicating a potential fault condition with respect to the IPG's operation or application of program parameters.

In some additional and/or alternative aspects, a system and method of operating an IPG may involve determining or selecting a discharge mode based on the measured VRES_{MEAS} compared against suitable thresholds. In an example arrangement, a method of operating the IPG including adjustable discharge parameters is disclosed. The IPG may include one or more leads implanted proximate to a patient's tissue, wherein each lead includes a plurality of electrodes, and the method may comprise applying one or more stimulation pulses to at least one electrode of the lead system; measuring a steady state residual voltage across the at least one electrode in reference to at least another electrode prior to delivery of a next stimulation pulse to the tissue; and modifying, responsive to the steady state residual voltage measurement, at least one of a discharge pulse amplitude and a discharge pulse width for generating a discharge pulse in an active discharge process operable to discharge the residual voltage to a pre-set value. In one variation, the method may further comprise effectuating a passive discharge operation following the active discharge process. In one variation, the active discharge process may be effectuated in response to a determination that the stead state residual voltage is greater than or equal to a threshold. In one variation, the pre-set value to which the residual voltage is discharged via active discharge may be based at least in part on a time period available for a passive discharge process during an inter-pulse interval.

Example embodiments of the present patent disclosure may be advantageously configured to obtain in vivo ETI parametric data as well as residual voltage data, which may be utilized in effectuating adaptive adjustment of a target therapy setting, thereby facilitating optimization of stimulation therapy provided to the patient. In some examples, by leveraging stimulation parameter ramping during therapy initialization and/or deactivation, which may be typically employed to mitigate unpleasant “shocking” sensations for the patient, the diagnostic information can be obtained without interference in the normal delivery of therapy to the patient. Moreover, optimization of therapy settings according to some embodiments herein can ensure patient safety and electrode integrity, minimize patient discomfort, as well as help monitor lead electrode integrity and extend the device battery longevity, thereby resulting in improved device performance.

Additional/alternative features and variations of the embodiments and associated benefits will be apparent in view of the following description and accompanying Figures.

### BRIEF DESCRIPTION OF THE DRAWINGS

Embodiments of the present disclosure are illustrated by way of example, and not by way of limitation, in the Figures of the accompanying drawings in which like references indicate similar elements. It should be noted that different references to “an” or “one” embodiment in this disclosure are not necessarily to the same embodiment, and such references may mean at least one. Further, when a particular feature, structure, or characteristic is described in connection with an embodiment, it is submitted that it is within the knowledge of one skilled in the art to effectuate such feature, structure, or characteristic in connection with other embodiments whether or not explicitly described.

The accompanying drawings are incorporated into and form a part of the specification to illustrate one or more exemplary embodiments of the present disclosure. Various advantages and features of the disclosure will be understood from the following Detailed Description taken in connection with the appended claims and with reference to the attached drawing Figures in which:

FIG. 1A depicts an example biostimulation system wherein one or more embodiments of an IMD diagnostic monitoring scheme may be practiced in accordance with the teachings herein;

FIG. 1B depicts an IMD having a conventional lead conductor arrangement including DC blocking capacitors according to an example arrangement;

FIG. 1C depicts a pulse generator portion having diagnostic circuitry configured with residual voltage (VRES) monitoring functionality wherein a lead conductor arrangement without DC blocking capacitors may be provided according to an embodiment of the present disclosure;

FIG. 1D depicts a generalized electrode/tissue interface (ETI) equivalent circuit arrangement for an IMD's lead electrode system that may be characterized for purposes of some examples of the present disclosure;

FIGS. 2A-2J depict representative lead and electrode arrangements that may be deployed in association with an IMD having VRES-based diagnostic monitoring functionality according to some examples of the present disclosure;

FIG. 3 depicts a diagnostic circuit portion that may be configured to effectuate VRES measurements for purposes of some examples of the present disclosure;

FIG. 4 depicts a VRES-based diagnostic monitoring scheme for effectuating discharge mode selection and DC fault detection according to some examples of the present disclosure;

FIGS. 5-7 depict flowcharts illustrative of blocks, steps, functions and/or acts that may be (re)combined in one or more arrangements for effectuating a VRES-based diagnostic monitoring scheme and/or providing a medical therapy to a patient according to some examples of the present disclosure;

FIG. 8 depicts a flowchart illustrative of blocks, steps, functions and/or acts that may be (re)combined in one or more arrangements with other flowcharts according to some examples of the present disclosure;

FIGS. 9-11 depict example VRES distributions or trends that may be used for diagnostic purposes according to some examples of the present disclosure;

FIG. 12 illustrates a representative spinal cord stimulation (SCS) therapy application involving an IPG/IMD and associated lead system having a plurality of electrodes wherein a VRES-based diagnostic monitoring scheme may be implemented according to an example embodiment of the present disclosure;

FIG. 13 depicts a relationship between predicted VRES and the electrode surface area in a stimulation setting scenario that may be used for diagnostic monitoring purposes, taken alone or in conjunction with one or more example diagnostic monitoring schemes of the present disclosure;

FIG. 14 depicts an example pulse train including an active discharge pulse having programmable/adjustable discharge pulse width and/or discharge pulse amplitude that may be effectuated according to some embodiments of the present disclosure; and

FIGS. 15A and 15B depict example pulse signal traces wherein a passive discharge scheme may be interspersed with an active discharge pulse consuming less battery power in some embodiments of an IMD according to the teachings herein.

### DETAILED DESCRIPTION

In the description herein for embodiments of the present disclosure, numerous specific details are provided, such as examples of circuits, devices, components and/or methods, to provide a thorough understanding of embodiments of the present disclosure. One skilled in the relevant art will recognize, however, that an embodiment of the disclosure can be practiced without one or more of the specific details, or with other apparatuses, systems, assemblies, methods, components, materials, parts, and/or the like. In other instances, well-known structures, materials, or operations are not specifically shown or described in detail to avoid obscuring aspects of embodiments of the present disclosure. Accordingly, it will be appreciated by one skilled in the art that the embodiments of the present disclosure may be practiced without such specific components. It should be further recognized that those of ordinary skill in the art, with the aid of the Detailed Description set forth herein and taking reference to the accompanying drawings, will be able to make and use one or more embodiments without undue experimentation.

Additionally, terms such as “coupled” and “connected,” along with their derivatives, may be used in the following description, claims, or both. It should be understood that these terms are not necessarily intended as synonyms for each other. “Coupled” may be used to indicate that two or more elements, which may or may not be in direct physical or electrical contact with each other, co-operate or interact with each other. “Connected” may be used to indicate the establishment of communication, i.e., a communicative relationship, between two or more elements that are coupled with each other. Further, in one or more example embodiments set forth herein, generally speaking, an electrical element, component or module may be configured to perform a function if the element may be programmed for performing or otherwise structurally arranged to perform that function.

Some embodiments described herein may be particularly set forth in the context of an implantable pulse generator (IPG) for generating electrical stimulation for application to a desired area of a body or tissue based on a suitable stimulation therapy application, such as a spinal cord stimulation (SCS) system. However, it should be understood that example circuitry and methods of operation disclosed herein are not limited thereto, but have broad applicability, including but not limited to different types of implantable devices such as neuromuscular stimulators and sensors, dorsal root ganglion (DRG) stimulation systems, deep brain stimulation (DBS) systems, cochlear implants, retinal implants, implantable cardiac rhythm management (CRM) devices, implantable cardioverter defibrillators, pacemakers, and the like, as well as implantable drug delivery/infusion systems, implantable devices configured to effectuate real-time measurement/monitoring of one or more physiological functions of a patient's body, including various implantable biomedical/bioelectrical sensors and sensing systems, wherein the foregoing devices and/or systems may be generally referred to as “biostimulation/biosensing systems”. Example circuitry and methods of operation disclosed herein are not limited to use with respect to an IPG or any particular form of IPG. For example, some embodiments may be implemented with respect to a fully implantable pulse generator, a radio frequency (RF) pulse generator, and/or a micro-implantable pulse generator, inter alia.

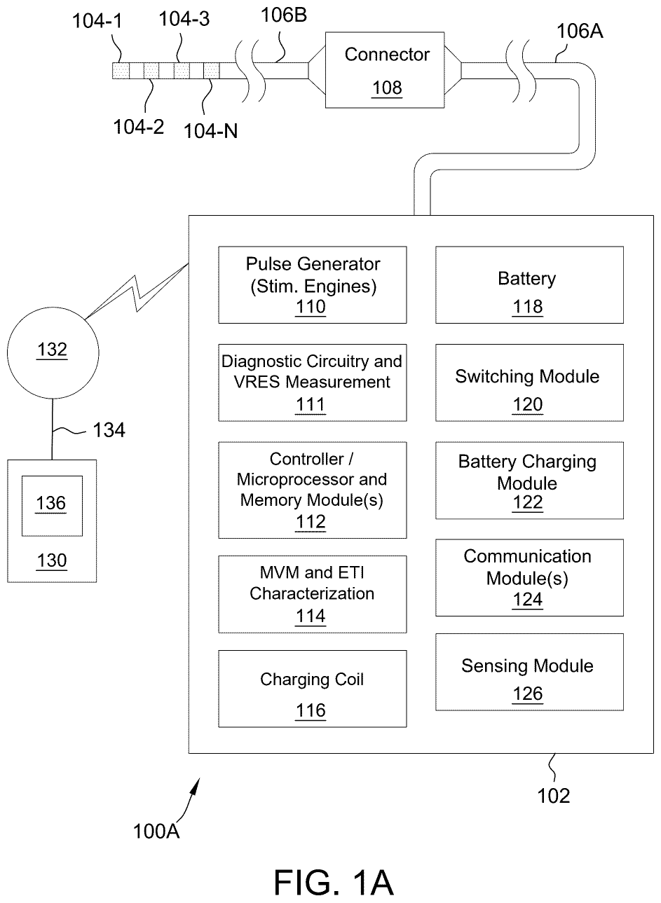

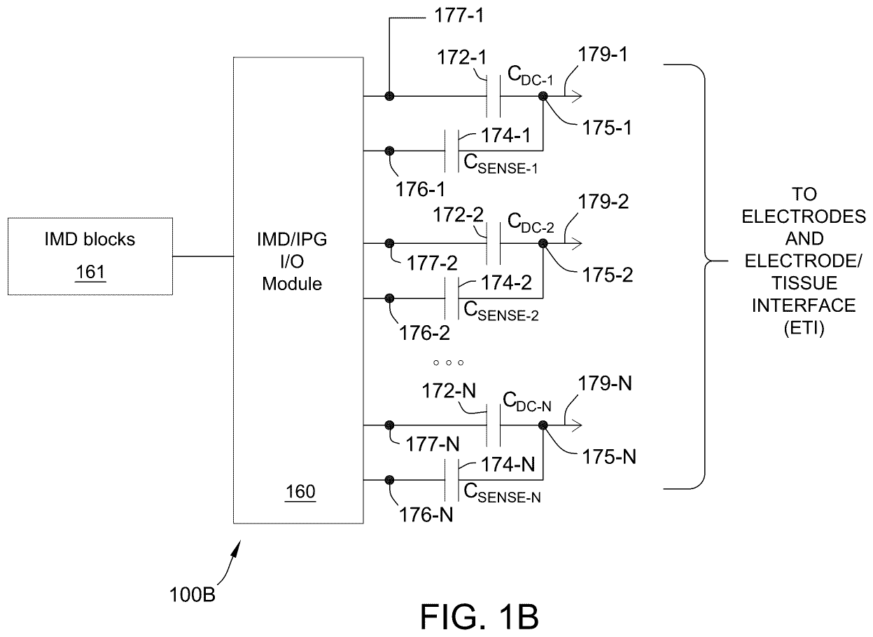

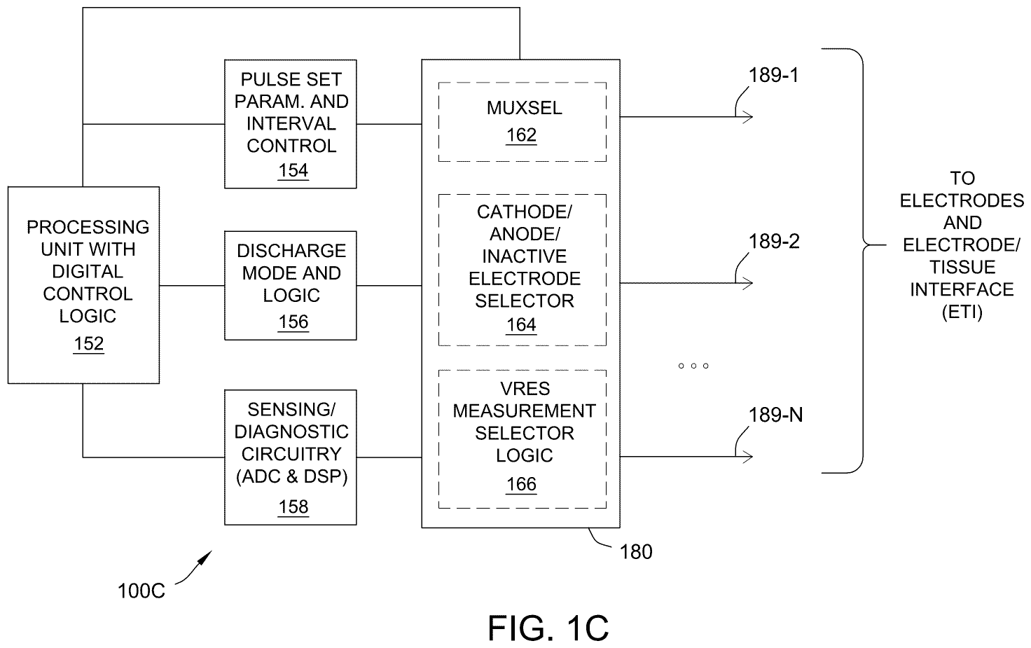

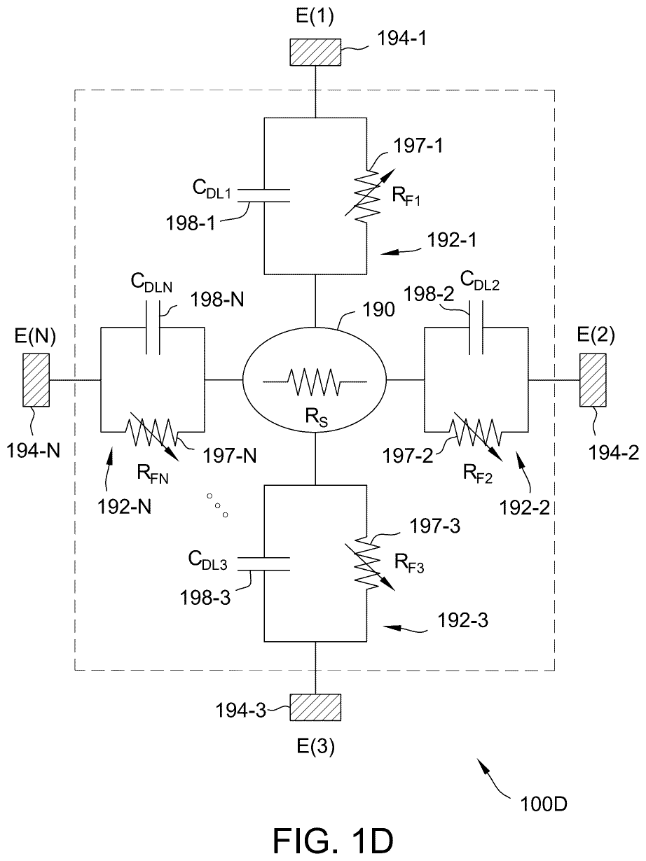

Referring to FIG. 1A, depicted therein is IMD system 100A where one or more embodiments of a diagnostic monitoring scheme of the present patent disclosure may be practiced according to the teachings herein. As will be seen in detail further below, example embodiments may involve effectuating in vivo voltage measurements including residual voltage (VRES) measurements as well as utilizing electrical load parameters of an electrode/tissue interface (ETI) equivalent circuit arrangement associated with the IMD's lead electrodes for a variety of diagnostic purposes such as, e.g., DC leakage fault detection, compliance with a stimulation therapy's safe operating areas, discharge mode selection, etc. By way of illustration, system 100A may be adapted to stimulate spinal cord tissue, peripheral nerve tissue, deep brain tissue, DRG tissue, cortical tissue, cardiac tissue, digestive tissue, pelvic floor tissue, or any other suitable biological tissue of interest within a patient's body, as noted above. System 100A includes an implantable pulse generator (IPG) or IMD 102 that comprises a diagnostic circuit and measurement module 111 adapted to effectuate various diagnostic operations based on electrical measurements, e.g., complex impedance measurements, residual voltage (VRES) measurements etc., that may be obtained with respect to one or more stimulated electrodes of a lead system coupled to the IMD, as will be set forth in additional detail further below. In one example embodiment, IPG 102 may be implemented as having a metallic housing or can that encloses a controller/processing block and associated memory, collectively shown as module(s) 112, pulse generating circuitry 110 that may comprise one or more stimulation engines operative to generate stimulation pulses based on suitable stimulation programs, a charging coil 116, a power supply such as e.g., battery 118, a far-field and/or near field communication block or module 124, battery charging circuitry 122, switching circuitry 120, sensing circuitry 126, and the like. Controller/processor module 112 may include one or more microcontrollers or other suitable processors for controlling the various components of IPG/IMD 102 under suitable programmatic control. Software/firmware code may be stored in one or more memory components of IPG 102, which may be integrated with the controller/processor module 112, and/or other suitable application-specific storage components for execution by the microcontroller or processor 112 and/or other programmable logic blocks to control the various components of the device for purposes of an embodiment of the present patent disclosure, as well as execute one or more methods herein as will be set forth in detail further below. For example, IMD 102 may additionally and/or alternatively include one or more nonvolatile memory (NVM) modules 114 for storing stimulation programs and/or program code configured to effectuate ETI parametric characterization and the estimation of expected VRES values for a given stimulation program. Depending on implementation, at least some portions of NVM 114 may be executed in conjunction with other modules of IMD 102 for effectuating diagnostic monitoring operations in some example embodiments.

In one arrangement, IPG 102 may be coupled to a separate or an attached extension component 106A for providing electrical and physical connectivity to an implantable lead 106B via a lead connector 108, wherein a distal end of the lead 106B includes a plurality of electrodes 104-1 to 104-N that may be selectively energized. Where the extension component 106A is provided as a separate component, the extension component 106A may connect with a “header” portion of IPG 102 as is known in the art. If the extension component 106A is integrated with IPG 102, internal electrical connections may be made through respective conductive components. In general, electrical pulses may be generated by the pulse generating circuitry 110 under the control of processing block 112, which may be provided to the switching circuitry 120 that is operative to selectively connect to electrical outputs of the IPG device, which are ultimately coupled to the electrodes 104-1 to 104-N at a distal end of the lead system 106B via respective electrical conductive traces.

In one arrangement, lead electrodes 104-1 to 104-N may be positioned along an axis of the lead 106B, with an angular offset such that the lead electrodes 104-1 to 104-N do not overlap. The lead electrodes 104-1 to 104-N may be in the shape of a ring such that each lead electrode continuously covers the circumference of the exterior surface of the lead 106B. Typically, the lead electrodes 104-1 to 104-N are separated from each other by non-conducting portions of the lead 106B, which electrically isolate each lead electrode 104-1 to 104-N from an adjacent lead electrode 104-1 to 104-N. The non-conducting portions of the lead 106B may include one or more insulative materials and/or biocompatible materials to allow the lead 106B to be implantable within the patient. Non-limiting examples of such materials include polyimide, polyetheretherketone (PEEK), polyethylene terephthalate (PET) film (also known as polyester or Mylar), polytetrafluoroethylene (PTFE) (e.g., Teflon), or parylene coating, polyether bloc amides, polyurethane, or the like compositions.

Additionally or alternatively, electrodes 104-1 to 104-N may be in the shape of a split or non-continuous ring such that the stimulation pulse(s) may be emitted in a manner so as to create an electric field emanating in an outward radial direction adjacent to the lead electrodes 104-1 to 104-N. Examples of lead electrodes 104-1 to 104-N and associated fabrication processes are disclosed in one or more of the following: (i) U.S. Patent Application Publication No. 2011/0072657, entitled, “METHOD OF FABRICATING STIMULATION LEAD FOR APPLYING ELECTRICAL STIMULATION TO TISSUE OF A PATIENT”; and (ii) U.S. Patent Application Publication No. 2018/0008821, entitled, “IMPLANTABLE THIN FILM DEVICES”, each of which is incorporated herein by reference.

It should be noted the lead electrodes 104-1 to 104-N may be in various other formations, for example, in a planar formation, in an array or grid, etc. on a paddle structure as disclosed in U.S. Patent Application Publication No. 2014/0343564, entitled, “PADDLE LEADS FOR NEUROSTIMULATION AND METHOD OF DELIVERING THE SAME”, which is incorporated herein by reference.

In one arrangement, the lead system 106B (as well as extension 106A where provided) may comprise a lead body of insulative material encapsulating a plurality of conductors within the material that extend from a proximal end (that is proximate to IPG 102) to the distal end of the lead body containing the lead electrodes 104-1 to 104-N. The conductors or conductive traces are operative to electrically couple the lead electrodes 104-1 to 104-N to a corresponding plurality of terminals (not shown) of the lead system 106A/B. In general, the terminals are adapted to receive electrical pulses from the pulse generation and switching circuitry of IPG 102, which are propagated via the corresponding conductive traces to at least a portion of the lead electrodes 104-1 to 104-N that are adapted to apply the pulses to a desired stimulation target of the patient depending on the particular stimulation therapy application. Also, sensing of physiological or bioelectrical signals may occur through the lead electrodes 104-1 to 104-N, corresponding conductors, and associated terminals in some additional and/or alternative embodiments. By way of illustration, an example embodiment of the stimulation system 100A may be provided with a plurality of lead electrodes 104-1 to 104-N comprising four electrodes, eight electrodes, etc., although any suitable number of electrodes (as well as corresponding conductive traces and terminals) may be provided in a lead system. Additionally or alternatively, various sensors (e.g., a position detector, temperature sensor, one or more electrochemical sensors, a radiopaque fiducial, etc.) may be located near the distal end of the lead 106B and electrically coupled to terminals through associated conductors within the lead body.

Although not required for any particular embodiment, the lead body of the implantable lead system 106A/106B may be fabricated in a manner that facilitates flexing and/or elongation upon implantation or advancing within or relative to the tissue (e.g., nervous tissue) of the patient towards the stimulation target to account for movement of the patient during or after implantation. Fabrication techniques and material characteristics for “body compliant” leads are disclosed in greater detail in U.S. Pat. No. 9,844,661, entitled “COMPLIANT ELECTRICAL STIMULATION LEADS AND METHODS OF FABRICATION”, which is incorporated herein by reference.

An example implementation of the components within IPG 102, such as, e.g., processor and associated charge control circuitry for an IPG, is described in U.S. Pat. No. 7,571,007, entitled “SYSTEMS AND METHODS FOR USE IN PULSE GENERATION”, which is incorporated herein by reference. An example implementation of circuitry for recharging a rechargeable battery (e.g., battery charging circuitry 122) of an IPG using inductive coupling and external charging circuits is described in U.S. Pat. No. 7,212,110, entitled “IMPLANTABLE DEVICE AND SYSTEM FOR WIRELESS COMMUNICATION”, which is incorporated herein by reference. Still further, an example implementation of “constant current” pulse generating circuitry (e.g., comprising at least a portion of pulse generating circuitry 110) is provided in U.S. Patent Application Publication No. 2006/0170486 entitled “PULSE GENERATOR HAVING AN EFFICIENT FRACTIONAL VOLTAGE CONVERTER AND METHOD OF USE”, which is incorporated herein by reference. One or multiple sets of such circuitry may be provided within IPG 102 operating in association with a current control module for providing stimulation across a select number of electrodes. Different stimulation pulses on different lead electrodes selected from electrodes 104-1 to 104-N may be generated using consecutively generated pulses according to a “multi-stimset program” as is known in the art. Complex pulse parameters may be employed such as those described in U.S. Pat. No. 7,228,179, entitled “METHOD AND APPARATUS FOR PROVIDING COMPLEX TISSUE STIMULATION PATTERNS”, and International Patent Publication Number WO 2001/093953, entitled “NEUROMODULATION THERAPY SYSTEM”, which are incorporated herein by reference. Alternatively, multiple sets of such stimulation circuitry may be employed to provide high frequency pulse patterns (e.g., tonic stimulation waveform, burst stimulation waveform, and the like) that may be generated and delivered for stimulation therapy through one or more leads 104-1 to 104-N as is also known in the art. Various sets of parameters may define the pulse characteristics and pulse timing for the pulses applied to the various lead electrodes as is known in the art. Although constant current pulse generating circuitry is contemplated for some embodiments, any other suitable type of pulse generating circuitry may be employed such as constant voltage pulse generating circuitry.

In an example implementation of IPG 102, sensing circuitry 126 may be provided, preferably adapted to measure a suitable electric parameter or transduced characteristic (e.g., voltage, current, capacitance, etc.), over a configurable period of time, associated with the stimulation target or tissue through at least one of the electrodes proximate to the stimulation target, e.g., electrodes configured to operate as biosensing inputs, wherein such “sensing” electrodes may be coupled to the sensing circuitry 126 via suitable alternating current (AC)-coupling capacitors as will be set forth further below. In an example embodiment, the sensing circuitry 126 may measure an evoked compound activation potential (ECAP) waveform from an Aβ sensory fiber or spinal cord. Optionally, the sensing circuitry 126 may be configured to store the measured/sensed electric data in memory 114. Furthermore, the diagnostic circuitry 111 may be configured to interoperate with the sensing circuitry 126 and pulse generation and switching functionalities of the IPG device 102 for effectuating diagnostic voltage measurements as well as in vivo electrical load characterization of one or more stimulated electrodes of the implanted lead system in some example embodiments.

An external device 130 may be implemented to charge/recharge the battery 118 of IPG 102 (although a separate recharging device could alternatively be employed), to access memory 114, and/or to program or reprogram IPG 102 with respect to the stimulation set parameters including pulsing specifications, ramping sequences, etc., while implanted within the patient. In alternative embodiments, however, separate programmer devices may be employed for charging and/or programming the IPG 102 device and/or any programmable components thereof. An example embodiment of the external device 130 may be a processor-based system having wireline and/or wireless communication capabilities, and may comprise a commercial off-the-shelf (COTS) equipment such as a portable computer, smartphone, smart pad, tablet, phablet, laptop, a personal digital assistant (PDA), or any smart wearable device and smart digital assistant device or the like, or a proprietary portable medical/healthcare device, which may be configured to execute a suitable therapy application program or “app” under programmatic control. Software may be stored within a non-transitory memory of the external device 130, which may be executed by the processor to control the various operations of the external device 130. A connector or “wand” 134 may be electrically coupled to the external device 130 through suitable electrical connectors (not specifically shown), which may be electrically connected to a telemetry component 132 (e.g., inductor coil, RF transceiver, etc.) at the distal end of wand 134 through respective communication links that allow bi-directional communication with IPG 102. Optionally, in some embodiments, the wand 134 may comprise one or more temperature sensors for use during charging operations.

In one general scenario, a user (e.g., a doctor, a medical technician, or the patient) may initiate communication with IPG 102 by placing the wand 134 proximate to the stimulation system 100A. Preferably, the placement of the wand 134 allows the telemetry system to be aligned with the far-field and/or near field communication circuitry 124 of IPG 102. The external device 130 preferably provides one or more user interfaces 136 (e.g., touch screen, keyboard, mouse, buttons, scroll wheels or rollers, or the like), allowing the user to operate IPG 102. The external device 130 may be controlled by the user through the user interface 136, allowing the user to interact with IPG 102, including, e.g., dynamically configuring electrodes for effectuating different measurement connection schemes, effectuating programmatic control for facilitating voltage measurements and extraction of electrical load parameters based on applicable equivalent ETI circuit models, etc., in addition to effectuating one or more corrective actions responsive to diagnostic indicators, as will be set forth further below. Further, the user interface 136 may permit the user to move electrical stimulation along and/or across one or more of the lead(s) 106A using different lead electrode combinations selected from electrodes 104-1 to 104-N, for example, as described in U.S. Patent Application Publication No. 2009/0326608, entitled “METHOD OF ELECTRICALLY STIMULATING TISSUE OF A PATIENT BY SHIFTING A LOCUS OF STIMULATION AND SYSTEM EMPLOYING THE SAME”, which is incorporated herein by reference. Optionally, the user interface 136 may permit the user to designate which electrodes 104-1 to 104-N are to stimulate (e.g., emit/sink current pulses, in an anode state or in a cathode state), or not selected to stimulate (i.e., remain inactive or floating, i.e., “unused”), with respect to a potential stimulation target, to measure/sense tissue electrical parameters, or the like. As used herein “stimulation” refers to the application of an electrical signal with respect to a target body tissue, regardless of the direction of current flow and/or effect that signal is intended to produce. Additionally or alternatively, the external device 130 may access or download the electrical measurements from the memory 114 acquired by the sensing circuitry 126 and/or diagnostic circuitry 111.

In some implementations, the external device 130 may permit operation of IPG 102 according to one or more spinal cord stimulation (SCS) programs or therapy applications to treat the patient. Each SCS program may include one or more sets of stimulation parameters of the pulse including pulse amplitude, stimulation level, pulse width, pulse frequency or inter-pulse period, pulse repetition parameter (e.g., number of times for a given pulse to be repeated for respective stimulation sets or stimsets during execution of program), biphasic pulses, monophasic pulses, etc. IPG 102 may be configured to modify its internal parameters in response to the control signals from the external device 130 to vary the stimulation characteristics of the stimulation therapy transmitted through the lead system 106A/106B to the tissue of the patient. Example neurostimulation (NS) systems, stimsets, and multi-stimset programs are set forth in U.S. Pat. No. 7,228,179, entitled “METHOD AND APPARATUS FOR PROVIDING COMPLEX TISSUE STIMULATION PATTERNS”, and International Patent Publication Number WO 2001/093953, entitled “NEUROMODULATION THERAPY SYSTEM”, which are incorporated hereinabove by reference.

Furthermore, one skilled in the art will recognize that although example lead system 106A/B shown in FIG. 1A is illustrated with a single implantable lead, the teachings herein are not necessarily limited thereto and an example embodiment of the present invention may involve a lead system comprising two or more implantable leads, with each lead having a plurality of electrodes, wherein different electrodes may be grouped into different channels in a stimulation application and a stimulation current may be split and mapped across a number of electrodes regardless of the channels or whether one or more leads are selected for stimulation. Accordingly, different electrode combinations may be selected for stimulation depending on a given stimulation program in an example embodiment, wherein applicable equivalent ETI circuit models may be obtained and used for VRES estimates in accordance with the teachings herein.

Providing a stimulation signal to a target body tissue, an accumulation of continuous or net charge at the electrode/tissue interface may occur, resulting in a residual voltage (VRES), which may not only dynamically affect the electrical characteristics of stimulation pulses being applied (e.g., resulting in increased battery current in some current-controlled stimulators) but can also contribute to deterioration of lead electrode integrity if not kept within particular bounds, as noted in the present disclosure. Further, because stimulation involves applying an electrical charge to body tissue, IMDs are required to ensure that a residual voltage at the ETI corresponding to each electrode is maintained within certain thresholds. Preferably, net charge at the ETI of an electrode needs to be as close to zero as possible, i.e., the stimulation is charge balanced. To maintain charge balance even under fault conditions, accordingly, some IPGs may include output coupling capacitors between the output circuits of the pulse generation/switching circuitry and the electrodes to block errant continuous direct current (DC) flows, wherein these capacitors as well as a double-layer capacitance or C_{DL} of the ETI (described further below) are discharged via “passive” charge balancing by shorting the stimulation electrodes together. In such arrangements, charge built up on the electrodes during stimulation may be offset by use of the output coupling capacitors (referred to herein as “DC blocking capacitors”) and may be discharged when delivery of a portion of the electrical signal is completed, e.g., typically after delivery of an individual pulse in a stimulation signal. A “discharge phase” may be observed for a period, for example, after a monophasic stimulation phase. In one arrangement, the stimulation phase and the discharge phase taken together may be considered a charge-balanced pulse in a signal comprising a plurality of such pulses. Even in such arrangements, however, there may be a gradual buildup of residual voltage across the DC blocking capacitors over time, depending on the frequency and type of pulsing schemes and associated stimsets used, in addition to the charge/voltage buildup at the ETI of an implantable lead system. As will be seen in detail further below, whereas some level of VRES is expected and allowed in passive discharge systems, example embodiments of the present disclosure may be advantageously configured to ensure that an applied stimulation program is operating in a manner such that VRES is within the permissible bounds for a given therapy scenario.

In general, ensuring stimulation safety in IMDs devices involves verifying that stimulation pulses are delivered to patients without causing discomfort or other deleterious effects, complying with applicable regulatory requirements, etc. A combination of hardware mitigations and conservative stimulation protocols may be implemented to achieve stimulation safety. However, such practices tend to be suboptimal in that there may be overdesign of IPG hardware as well as extra guard-banding of rules governing what types of stimulation can be safely delivered to patients over a range of physiological states, activity modes, etc. For example, some existing solutions involve maximizing the DC blocking capacitors on all stimulator output channels, selecting discharge modes based on nominal impedance values, and utilizing stimulation parameter settings and/or thresholds long known to be safe, and the like. Limits on safe amounts of charge delivered and charge density are also factored in setting tolerances, which makes programming more restrictive than necessary in some example arrangements.

Several deficiencies and shortcomings exist with respect to the foregoing techniques, however. For example, having large DC blocking capacitors (around 5 μF to 25 μF) on every channel significantly increases the circuit board area of an IMD, especially where the channel count is fairly high, for instance up to 32 or more in some applications. Further, applied stimulation rules must be overly conservative to cover worst-case corner combinations and use case scenarios. In addition, the rules are generically defined and not individualized or personalized to the particular patient and/or the lead types that are implanted. Relatedly, the rules are set in a fixed manner, e.g., at programming, and do not evolve or change with the patient over time depending on the patient's physiological state, daily activities, and the like. Still further, in typical IMD arrangements, safety of charge delivered per pulse and charge balance period are determined upfront based on the given programmed stimulation parameters instead of the actual stimulation being delivered to the patient.

Examples described herein recognize the foregoing deficiencies and shortcomings and accordingly provide a diagnostic monitoring scheme based on actual VRES measurements and estimated VRES determinations predicted from a known ETI equivalent circuit model for a given stimulation program, wherein various indications relating to potential fault conditions that may arise in a therapy setting may be detected prophylactically such that the need for baseline conservative practices and hardware mitigations may be obviated. For example, in some embodiments an IMD may be configured with a lead system without having to place DC blocking capacitors on every channel. Some examples may also be configured to provide a discharge mode selection scheme that may be configured in a dynamic manner based on VRES monitoring. It should be appreciated that while the examples of the present disclosure may be expected to provide various tangible improvements over baseline IMD implementations that aim to ensure stimulation safety, no particular result is a requirement unless explicitly recited in a particular claim appended hereto.

Turning to FIG. 1B, depicted therein is a baseline IMD 100B having a conventional lead conductor arrangement and associated DC blocking capacitors, which may be potentially eliminated in an example embodiment of the present disclosure as will be set forth in detail further below. Whereas functional blocks of the IMD 100B are collectively/generally shown as a single block 161 in this Figure for the sake of simplicity, an input/output (I/O) interface block 160 thereof is particularly exemplified herein as having a plurality of lead connectors 179-1 to 179-N coupled thereto, each lead connector representing a channel in an example arrangement. In general, lead connectors 179-1 to 179-N may be configured as part of a lead system having one or more leads (not specifically shown), which may be interfaced with respective electrodes and associated ETI portions that may be represented as circuitry based on known or heretofore unknown charge-transfer mechanisms or models (not shown in this Figure). Each lead connector 179-1 to 179-N may be provided with a DC blocking stimulation capacitor (C_{DC}) and an AC-coupling sense capacitor (C_{SENSE}) for facilitating direct current flow blocking and AC-coupling functionality with respect to the corresponding electrode that may be configured to operate as a stimulation node or a sensing node in an example implementation, although some lead system arrangements may be configured without AC-coupling sense capacitors. By way of illustration, DC blocking stimulation capacitor C_{DC-1} 172-1 and sense capacitor C_{SENSE-1} 174-1 are coupled to lead connector 179-1 such that two interface terminals 177-1 and 176-1 are effectuated with respect to the frontend lead circuitry of the interface block 160. Sense capacitor C_{SENSE-1} 174-1 is shunted across C_{DC-1} 172-1 such that an intermediate tap or node 175-1 is effectuated in association with the lead connector 179-1. Likewise, remaining lead connectors 179-N may be provided with respective C_{SENSE-N} 174-N capacitors shunted across respective C_{DC-N} 172-N capacitors to facilitate two interface terminals or nodes 177-N and 176-N for each corresponding lead electrode connector. Depending on the type of leads, which in turn may depend on the type of stimulation therapy application, example C_{DC} capacitances can be substantially larger than the capacitances associated with the complex impedance of the ETI equivalent circuitry (e.g., by several orders of magnitude) and may consume a significant portion of the IMD circuit board as previously mentioned.

FIG. 1C depicts a pulse generator portion having diagnostic circuitry including residual voltage (VRES) monitoring functionality and associated lead conductor arrangement, wherein DC blocking capacitors may be omitted according to an embodiment of the present disclosure. One skilled in the art will recognize upon reference hereto that various functionalities associated with example blocks shown as part of or otherwise associated with the illustrated pulse generator portion 100C may be distributed and/or integrated among one or more blocks, subsystems and/or modules described hereinabove with respect to FIG. 1A. Consistent with the description set forth previously, a processing unit or controller 152 having or associated with suitable digital control logic is operatively coupled to a pulse control module 154 operative to generate one or more pulses according to selected stimulation settings, a discharge module 156 operative according to a selected discharge mode, and sensing/diagnostic circuitry 158, which may be configured for facilitating various functionalities including but not limited to voltage measurements, active discharge cycling, electrode selection and configuration, etc., as well as electrical load characterization of an equivalent ETI circuit arrangement associated with a lead system under appropriate programmatic control. An I/O interface block 180 is operatively coupled to a plurality of lead connectors 189-1 to 189-N comprising a lead system interfaced with respective electrodes and associated ETI that may be represented as a lumped element circuit model as set forth below. Because DC blocking capacitors are eliminated in an example embodiment of the present disclosure, the lead connectors 189-1 to 189-N associated with the pulse generator portion 100C may be coupled to respective ETI portions without corresponding DC blocking capacitors, wherein the diagnostic circuitry 158 may be configured to effectuate appropriate DC fault detection capability in a prophylactic manner according to the teachings herein. Although the illustrated embodiment of FIG. 1C exemplifies an arrangement where the lead connectors are not specifically shown with corresponding sense capacitors, it should be appreciated that in some examples one or more lead connectors may be provided respective sense capacitors, e.g., with respect to the electrodes configured as sensing electrodes. Accordingly, skilled artisans will recognize that the example arrangement shown FIG. 1C is merely illustrative and various additional and/or alternative lead configurations may be realized within the scope of the present patent disclosure, with or without C_{DC} and/or C_{SENSE} capacitors, for purposes of some example embodiments set forth herein. It should therefore be appreciated that the teachings of the present disclosure may be practiced without these additional capacitances in some example implementations.

In one arrangement, interface block 180 may include appropriate multiplexing and selection (MUXSEL) circuitry 162, anode/cathode/inactive electrode configuration circuitry 164 and VRES measurement selection logic 166 for effectuating various operations with respect to at least a subset of the selected electrodes depending on the configuration. For example, different electrodes of a lead system may be selectively configured for stimulation (e.g., anodic or cathodic stimulation), sensing, or designating unused/inactive states, etc., with appropriate electrical connections being made within an IPG device accordingly relative to the various components therein, preferably under suitable programmatic control as needed according to some embodiments. Example diagnostic circuitry 158 may comprise suitable analog-to-digital converter (ADC) circuitry configured for digital voltage measurement and associated signal processing using a variety of voltage measurement techniques. As such, voltage measurement circuitry can be external and/or internal, on-board or off-board, and/or may be coupled to other measurement devices. In some arrangements, sensing/diagnostic circuitry 158 may be configured to monitor the residual voltage (VRES_{MEAS}) using one or more preconfigured thresholds and associated interrupts, or in a zero current (0 mA) impedance measurement that may be configured to enable a comparison against an arbitrary threshold. In some examples, sensing/diagnostic circuitry 158 may be configured to perform determining, measuring, computing or otherwise obtaining both the resistive and capacitive components of a complex impedance parameter associated with the ETI circuit model. In some arrangements, components such as a function generator, waveform generator circuitry, and a transimpedance current amplifier may be utilized in measuring an ETI's complex impedance. In some arrangements, the resistive and capacitive components of an ETI may be applied in a known VRES model to obtain expected or estimated VRES values (VRES_{EXP} or VRES_{EST}) for a given stimulation program as will be set forth in further detail below.

Depending on implementation, monitored VRES_{MEAS} may be used in a number of arrangements with respect to one or more following diagnostic scenarios, provided herein as non-limiting examples. In one arrangement, diagnostic circuitry 158 may be configured to interoperate with other modules of the IMD device to determine that the delivered stimulation parameters match the programmed stimulation parameters (e.g., within a predetermined degree of tolerance such as ±5%, ±10%, etc.). The diagnostic circuitry 158 may further be configured to determine that a mismatch between the monitored VRES_{MEAS} and the estimated VRES_{EST} obtained for a given stimulation regime may be attributable to certain potential fault scenarios arising in the delivery of stimulation. For example, a functional relationship between the VRES mismatch and the stimulation parametric mismatch may be obtained using correlation/regression techniques, numerical/statistical analysis, etc., which may be used as a diagnostic tool in some arrangements. Further, monitored VRES_{MEAS} may be used to select a suitable discharge mode in a discharge cycle (e.g., passive discharge, balanced active discharge, or duration of asymmetrical active discharge, etc.), and/or to ensure that the selected discharge mode is operating at maximum allowed settings without compromising the device's safe operating area (SOA). Still further, monitored VRES_{MEAS} and the trends therein may be used to detect potential DC fault conditions with respect to the device operation, which may be attributable to a leakage fault pertaining to one or more of the stimulated/sensed electrode(s), and/or another component of the IMD device. In additional and/or alternative arrangements, monitored VRES_{MEAS} and the trends therein may be used to detect, determine and/or otherwise ascertain that the implanted electrodes are functionally behaving in a suitable manner expected of the electrodes based on their design and/or therapy application.

In some arrangements, active charge balancing may be effectuated by applying a discharge pulse of opposite polarity at a select electrode to reduce or eliminate the individual residual voltages of select electrodes by using discharge cycle module 156 in conjunction with switch circuitry under suitable programmatic control. Additional details regarding configuring lead electrodes as cathodes or anodes, either during stimulation or for discharging, may be found in U.S. Patent Application Publication No. 2009/0048643, entitled “METHOD FOR PROVIDING MULTIPLE VOLTAGE LEVELS DURING PULSE GENERATION AND IMPLANTABLE PULSE GENERATING EMPLOYING THE SAME”, which is hereby incorporated herein by reference for purposes of some example embodiments.

As some of the foregoing example embodiments may be particularly set forth in the context of an ETI equivalent circuit representation associated with the stimulated electrodes, a brief description of ETI modeling is provided in the following sections. When an electrode is placed near biological tissue and energized, there is a flow of current depending on the flow of electrons in the electrode and flow of ions in the tissue. The electrode/electrolyte (i.e., tissue) interface (EEI or ETI; also sometimes referred to as electrode/patient interface or EPI) may be modeled in accordance with a lumped element charge transfer model (e.g., Randles equivalent circuit of the electrode-electrolyte interface), involving a series of lumped resistor elements coupled with a shunt capacitance that models the double layer of charge at the interface. FIG. 1D depicts a generalized ETI equivalent circuit arrangement 100D for an IMD's lead electrode system that may be characterized and/or utilized according to an embodiment of the present disclosure. In the illustrated arrangement 100D, the solution resistance, R_{S}, is representative of the bulk electrolyte, which models the tissue or patient resistance, R_{PATIENT}, as a pure resistive component 190 disposed across electrodes E(1) 194-1 to E(N) 194-N, e.g., between any pair or sets of electrodes disposed as part of a circuit. With respect to each electrode, a double-layer capacitance or C_{DL} models the double layer of charge at the interface, which is coupled in parallel to a charge transfer resistance R_{CT}, also referred to as Faradaic resistance (R_{F}), across the interface. The resistive component comprising Faradaic resistance, R_{F}, disposed in parallel with the reactive component comprising the double-layer capacitance, C_{DL}, is operable as part of a complex impedance element that controls the conduction of charge through the interface, which can occur through various mechanisms, e.g., typically through oxidation-reduction reactions at the electrode for efficient operation of stimulation electrodes. Reference numerals 192-1 to 192-N shown in FIG. 1D accordingly refer to equivalent circuit representations of ETIs (or ETI portions) associated with corresponding electrodes 194-1 to 194-N, respectively, wherein C_{DL1} 198-1 to C_{DLN} 198-N and R_{F1} 197-1 to R_{FN} 197-N are illustrative of the respective lumped capacitive and resistive components thereof, which together represent respective complex impedances operative in the electrode-tissue interface environment of a patient. Whereas more complex models of the electrode/tissue interface may be used, the foregoing charge transfer model is illustrated herein without necessarily being limited thereto for purposes of facilitating individual electrode voltage measurements and electrical load characterization of the individual electrodes of a lead system.

In some arrangements, electrical load characterization of an ETI circuit representation associated with an electrode involves determining, estimating, obtaining or otherwise characterizing the resistive and the capacitive components of the complex impedance (Z) associated with the ETI circuit representation, wherein Z=R+iC and R is the bulk resistance (neglecting the effect of Faradaic resistance in normal conditions) and C is the double-layer capacitance of the ETI circuit. In the complex number domain, Z represents a phaser that defines the effective opposition to alternating current presented by the combined effect of ohmic resistance and reactance in a circuit that may be determined as an addition of the phaser that represents the current and the phaser that represents the voltage, which may have a fixed phase angle therebetween. In some arrangements, both the resistive and the capacitive components may be determined from in vivo or in situ voltage measurements over a course of time, which may be used in an example embodiment of the present disclosure for purposes of predicting VRES_{EST} values associated with an electrode, e.g., until a steady state is reached according to a known model.

In an example, the steady-state residual voltage can be estimated by iterating on the following equations until a steady-state is reached:

$$VPULSE = ( I ⋆ PW ) / C for build up during each pulse ,$$

where I=Amplitude, PW=Pulse Width, C=Double-layer Capacitance; and

$$VRES ( n ) = ( V R E S ( n - 1 ) + VPULSE ) * {e}^{( - t / RC )} ,$$

where t=time available for discharge, R=Bulk Resistance, C=Double-layer Capacitance and n=n^{th} pulse.

In an example, appropriate resistive (R) and capacitive (C) components may be determined using an ETI load characterization scheme set forth in U.S. Pat. No. 11,351,376, issued Jun. 7, 2022, entitled, “PARAMETRIC CHARACTERIZATION OF AN IMPLANTED STIMULATION LEAD SYSTEM ASSOCIATED WITH AN IMPLANTABLE PULSE GENERATOR,” which is incorporated by reference herein. Further, some example embodiments may involve measuring voltages using the measurement techniques described in U.S. patent application Ser. No. 16/195,502, filed Nov. 19, 2018, entitled, “KELVIN CONNECTION SCHEME FOR DIAGNOSTIC CAPABILITY IN A NEUROSTIMULATOR”, which is incorporated by reference herein. It should be appreciated by skilled artisans, however, that the foregoing techniques are not necessary for practicing an example embodiment of the present invention. Accordingly, various other known or heretofore unknown schemes for ETI load characterization and voltage measurements may be utilized for purposes of the example embodiments set forth herein.

In an example arrangement, a system and method may be configured to use VRES measurements and predicted VRES estimates as a broad safety indicator with respect to one or several aspects of IMD device operations. In one implementation, the IMD device may be provided with the circuitry and associated firmware/software configured to perform the following acts: (i) measuring, determining or otherwise obtaining complex impedances associated with respective ETI portions; (ii) separating the resistive and reactive/capacitive components respectively; (iii) periodically (or at configured times) measuring the steady state residual voltage using an ADC or comparator circuit; (iv) utilizing the stimulation parameters in an ETI-VRES model; and (v) predicting the expected VRES values for given stimulation settings. In further arrangements, techniques such as expert systems, artificial intelligence (AI), machine learning (ML), pattern recognition, etc., may be implemented to identify, detect or other recognize certain specific patterns in the VRES distributions indicative of potential fault conditions. For example, an unbounded linear increase in VRES measurements over a number of applied pulses is indicative of DC leakage, which is a designated fault condition that may be caused by a faulty electrode or other components of the device as previously mentioned. Some AI/ML-based techniques may also be configured to identify or determine (i) a configurable residual voltage threshold for switching from passive to active discharge, (ii) a configurable residual voltage threshold for determining how much reverse current to deliver for active discharge; (iii) whether the residual voltages or steady state distributions match expected patterns for the particular programmed parameters and/or implanted electrodes.

It will be appreciated that optimal stimulation settings for an individual patient therapy application may vary over time and may require taking into account the dynamic variability typically present in any biostimulation therapy due to, e.g., the variation of capacitive components implemented in a lead system, electrophysiological/electrochemical variability within a patient tissue over time as well as across different patients (i.e., inter-patient variability), in addition to the temporal variability of in situ and/or in vivo electrical characteristics of the implanted electrodes, and the like. Accordingly, it becomes desirable to accurately characterize the electrical loads or impedances presented by the IPG lead system under in situ and/or in vivo conditions such that a particular therapy setting may be dynamically modified, modulated, or otherwise adjusted to an optimized setting based on the real-time parameterization of the electrical loads modeled by an equivalent ETI circuit arrangement as set forth above. Further, it is also advantageous to obtain an IPG's electrical load information in real-time without unduly interfering with a normal stimulation program or protocol implemented for an individual patient therapy application as well as without incurring extra cost in terms of processing, battery power, etc.

As set forth the present disclosure, various types of electrodes having different form factors may be utilized based on the therapy application, each of which may exhibit different ETI characterizations depending on the form factor, e.g., shape, size, orientation, etc. For example, in a typical DBS implementation where segmented ring electrodes may be utilized, C_{DL} capacitances associated with the stimulated electrodes may be in the range of nanofarads (nF) whereas the C_{DL} capacitances associated with SCS electrodes may be in the range of microfarads (μF). Because the expected VRES for different types of electrodes are predicted to exhibit certain distributions, a deviation of a measured VRES distribution from an expected VRES distribution may be indicative of a potential fault condition, which may be identified or detected in an example embodiment of the present disclosure.

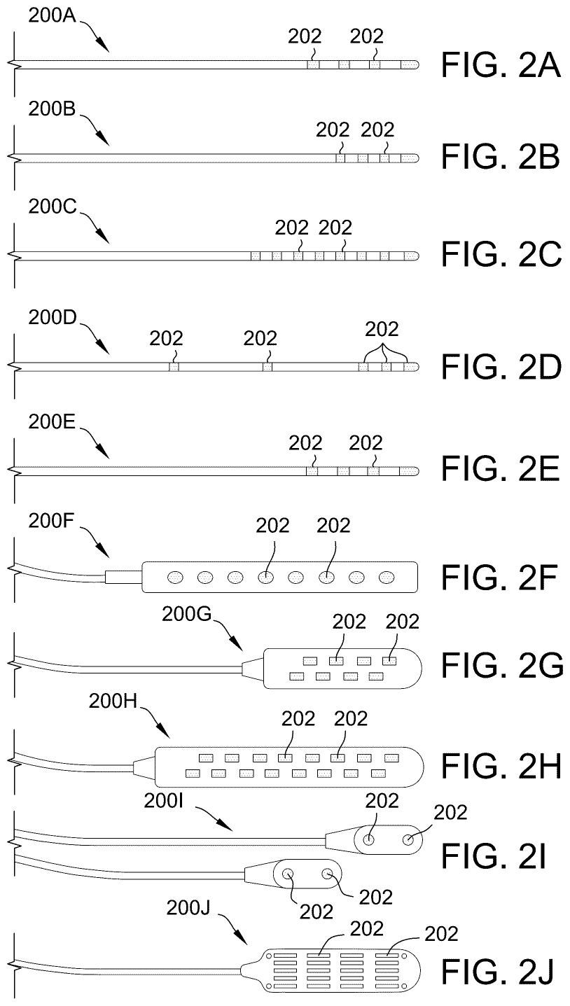

FIGS. 2A-2J depict representative lead and electrode arrangements that may be deployed in association with an IMD having VRES-based diagnostic monitoring functionality according to some examples of the present disclosure. One or more stimulation leads 200A to 200J are exemplary of a variety of commercially available leads, such as deep brain leads, percutaneous leads, paddle leads, etc., as shown in FIGS. 2A-2J, respectively, wherein conductive electrodes can be planar electrodes, ring electrodes, segmented or split electrodes, etc., commonly shown as electrodes 202. The non-conducting portions of leads 200A-200J may comprise a variety of insulative materials and/or biocompatible materials that allow the lead to be implantable within the patient as noted previously.

An example lead having electrodes 202 may be implanted in a patient such that one or more stimulation electrodes 202 of each stimulation lead 200A-J are positioned or disposed near, adjacent to, directly on or onto, proximate to, directly in or into or within the target tissue or predetermined site of the patient. Techniques for implanting stimulation electrodes are well known by those of skill in the art and may be positioned in various body tissues and in contact with various tissue layers; for example, deep brain, cortical, subdural, subarachnoid, epidural, cutaneous, transcutaneous and subcutaneous implantation is employed in some embodiments.

By way of illustration, set forth below are exemplary tissues, regions and/or organs that may be stimulated by an IMD device having VRES-based diagnostic monitoring functionalities according to some embodiments:

### A) Brain:

Central neuronal tissue includes brain tissue, spinal tissue or brainstem tissue. Brain tissue can include the frontal lobe, the occipital lobe, the parietal lobe, the temporal lobe, the cerebellum, or the brain stem. More specifically, brain tissue can include subcortical targets, for example, thalamus/sub-thalamus (i.e., thalamic nuclei, medial and lateral geniculate body, intralaminar nuclei, nucleus reticularis, pulvinar, subthalamic nuclei (STN), etc.), basal ganglia (i.e., putamen, caudate nucleus, globus pallidus), hippocampus, amygdala, hypothalamus, epithalamus, mammillary bodies, ventral tegmental area (VTA), substantia nigra, corpus callosum, fornix, internal capsula, anterior and posterior commissural, cerebral peduncles etc. Brain tissue also includes cerebellum, cerebellar peduncles, and cerebellar nuclei such as fastigial nucleus, globose nucleus, dentate nucleus, emboliform nucleus. Still further, in addition to the above mentioned subcortical targets, brain tissue also includes cortical targets, for example, auditory cortex, prefrontal cortex, the dorsolateral prefrontal cortex, the ventromedial prefrontal cortex, the cingulate cortex, subcallosal area, anterior cingulate cortex, the subgenual anterior cingulate cortex, the motor cortex and the somatosensory cortex. The somatosensory cortex comprises the primary, the secondary somatosensory cortex, and the somatosensory association complex. Still further, the somatosensory cortex also includes Brodmann areas 1, 2, 3, 5, and 7. Yet further, brain tissue can include various Brodmann areas for example, but not limited to Brodmann area 9, Brodmann area 10, Brodmann area 24, Brodmann area 25, Brodmann area 32, Brodmann area 39, Brodmann area 41, Brodmann area 42, and Brodmann area 46.

While not being bound by the description of a particular procedure, patients who are to have an electrical stimulation lead or electrode implanted into the brain for deep brain stimulation, generally, first have a stereotactic head frame, such as the Leksell, CRW, or Compass, mounted to the patient's skull by fixed screws. Subsequent to the mounting of the frame, the patient typically undergoes a series of magnetic resonance imaging (MRI) sessions, during which a series of two dimensional slice images of the patient's brain are built up into a quasi-three dimensional map in virtual space. This map is then correlated to the three dimensional stereotactic frame of reference in the real surgical field. In order to align these two coordinate frames, both the instruments and the patient must be situated in correspondence to the virtual map. In some embodiments, a current way to do this is to rigidly mount the head frame to the surgical table. Subsequently, a series of reference points (e.g., fiducials) may be established to relative aspects of the frame and patient's skull, so that either a person or a computer software system can adjust and calculate the correlation between the real world of the patient's head and the virtual space model of the patient MRI scans. The surgeon is able to target any region within the stereotactic space of the brain with precision (e.g., within 1 mm). Initial anatomical target localization is achieved either directly using the MRI images or functional imaging (PET or SPECT scan, fMRI, MSI), or indirectly using interactive anatomical atlas programs that map the atlas image onto the stereotactic image of the brain. In some arrangements, the anatomical target(s) or predetermined site(s) may be stimulated directly or affected through stimulation in another region of the brain.

In addition to deep brain stimulation, cortical stimulation can also be used to stimulate various brain tissues. Any of the stimulation leads illustrated in FIGS. 2A-2J can be used for cortical stimulation, as well as any other cortical electrode or electrode array. For implanting conventional cortical electrodes, it typically requires a craniotomy under general anesthesia to remove a relatively large (e.g., thumbnail-sized or larger) window in the skull. A pilot hole (e.g., 4 mm or smaller) can be formed through at least part of the thickness of the patient's skull adjacent a selected or predetermined site. In certain embodiments, the pilot hole can be used as a monitoring site.

The location of the pilot hole (and, ultimately the electrode received therein) can be selected in a variety of fashions, for example, the physician may use anatomical landmarks, e.g., cranial landmarks such as the bregma or the sagittal suture, to guide placement and orientation of the pilot hole or the physician may use a surgical navigation system. Navigation systems may employ real-time imaging and/or proximity detection to guide a physician in placing the pilot hole and in placing the electrode in the pilot hole. In some systems, fiducials are positioned on the patient's scalp or skull prior to imaging and those fiducials are used as reference points in subsequent implantation. In other systems, real-time MRI or the like may be employed instead of or in conjunction with such fiducials. A number of suitable navigation systems are commercially available, as is known to one skilled in the art. Once the pilot hole is formed, the threaded stimulation lead may be advanced along the pilot hole until the contact surface electrically contacts a desired portion of the patient's brain. If the stimulation lead is intended to be positioned epidurally, this may comprise relatively atraumatically contacting the dura mater; if the electrode is to contact a site on the cerebral cortex, the electrode will be advanced to extend through the dura mater. Thus, the lead may be placed epidurally or subdurally for cortical stimulation in some therapy systems having waveform program generation, storage and playback.

B) Spinal Cord and/or Peripheral Nerves

Peripheral nerves can include, but are not limited to olfactory nerve, optic nerve, oculomotor nerve, trochlear nerve, trigeminal nerve, abducens nerve, facial nerve, vestibulocochlear (auditory) nerve, glossopharyngeal nerve, vagal nerve, accessory nerve, hypoglossal nerve, occipital nerve (e.g., suboccipital nerve, the greater occipital nerve, the lesser occipital nerve), the greater auricular nerve, the lesser auricular nerve, the phrenic nerve, brachial plexus, radial axillary nerves, musculocutaneous nerves, radial nerves, ulnar nerves, median nerves, intercostal nerves, lumbosacral plexus, sciatic nerves, common peroneal nerve, tibial nerves, sural nerves, femoral nerves, gluteal nerves, thoracic spinal nerves, obturator nerves, digital nerves, pudendal nerves, plantar nerves, saphenous nerves, ilioinguinal nerves, gentofemoral nerves, and iliohypogastric nerves. Furthermore, peripheral neuronal tissue can include but is not limited to peripheral nervous tissue associated with a dermatome.

Spinal tissue can include the ascending and descending tracts of the spinal cord, more specifically, the ascending tracts of that comprise intralaminar neurons or the dorsal column. For example, the spinal tissue can include neuronal tissue associated with any of the cervical vertebral segments (C1, C2, C3, C4, C5, C6, C7 and C8) and/or any tissue associated with any of the thoracic vertebral segments (T1, T2, T3, T4, T5, T6, T7, T8, T9, T10, T11, T12) and/or any tissue associated with any of the lumbar vertebral segments (L1, L2, L3, L4. L5, L6) and/or any tissue associated with the sacral vertebral segments (S1, S2, S3, S4, S5). More specifically, the spinal tissue is the dorsal column of the spinal cord. The brainstem tissue can include the medulla oblongata, pons or mesencephalon, more particular the posterior pons or posterior mesencephalon, Lushka's foramen, and ventrolateral part of the medulla oblongata.

In other embodiments, the stimulation leads may be positioned in communication with the neuronal tissue of the spinal cord, more specifically, the dorsal column of the spinal cord. For example, stimulation electrodes are commonly positioned external to the dura layer surrounding the spinal cord. Stimulation on the surface of the cord is also contemplated, for example, stimulation may be applied to the spinal cord tissue as well as to the nerve root entry zone. Stimulation electrodes may be positioned in various body tissues and in contact with various tissue layers; for example, subdural, subarachnoid, epidural, and cutaneous, and/or subcutaneous implantation is employed in some embodiments.

Spinal cord stimulation, e.g., by way of program record playback by a waveform player as set forth herein, can be accomplished utilizing either percutaneous leads and/or laminotomy type leads that comprise a paddle. Percutaneous leads commonly have two or more equally-spaced electrodes which are placed above the dura layer through the use of a Touhy-like needle. For insertion, the Touhy-like needle is passed through the skin between desired vertebrae to open above the dura layer.

In contrast to the percutaneous leads, laminotomy leads have a paddle configuration and typically possess a plurality of electrodes (for example, two, four, eight, sixteen or twenty) arranged in one or more columns. Implanted laminotomy leads are commonly transversely centered over the physiological midline of a patient. In such position, multiple columns of electrodes are well suited to address both unilateral and bilateral pain, where electrical energy may be administered using either column independently (on either side of the midline) or administered using both columns to create an electric field which traverses the midline. A multi-column laminotomy lead enables reliable positioning of a plurality of electrodes, and in particular, a plurality of electrode columns that do not readily deviate from an initial implantation position.

Laminotomy leads require a surgical procedure for implantation. The surgical procedure, or partial laminectomy, requires the resection and removal of certain vertebral tissue to allow both access to the dura and proper positioning of a laminotomy lead. The laminotomy lead offers a more stable platform, which is further capable of being sutured in place that tends to migrate less in the operating environment of the human body. Depending on the position of insertion, however, access to the dura may only require a partial removal of the ligamentum flavum at the insertion site. In some embodiments, two or more laminotomy leads may be positioned within the epidural space, and the leads may assume any relative position to one another.

In certain embodiments, the stimulation leads may be placed subcutaneously on the patient's head. For example, one or more stimulation leads can be implanted subcutaneously such that one or more stimulation electrodes are positioned in communication with a dermatome area, for example (C1, C2, C3, C4, C5, C6, C7, and C8), cervical nerve roots (e.g., C1, C2, C3, C4, C5, C6, C7 and C8) cranial nerves (e.g., olfactory nerve, optic, nerve, oculomotor nerve, trochlear nerve, trigeminal nerve, abducent nerve, facial nerve, vestibulocochlear nerve, glossopharyngeal nerve, vagal nerve, accessory nerve, and hypoglossal nerve) and/or occipital area. For example, one or more stimulation electrodes are positioned in the C2 dermatome area/C3 dermatome area, subcutaneously, but superior to the galea. Within certain areas of the C2 dermatome area or occipital or occiput area, there is little or no muscle, this area primarily consists of fat, fascia, periosteum, and neurovascular structures (e.g., galea). More specifically, the electrode can be implanted in a subcutaneous fashion such that the electrode is positioned below the skin, above the bone on the back of the head or superior to the periosteum. On the back of the head, the probe is positioned in the C2 dermatome area or positioned at the back of the patient's head at about the level of the ear.

### C) Brainstem Stimulation

Implantation of a stimulation lead in communication with the predetermined brainstem area can be accomplished via a variety of surgical techniques that are well known to those of skill in the art. For example, an electrical stimulation lead can be implanted on, in, or near the brainstem by accessing the brain tissue through a percutaneous route, an open craniotomy, or a burr hole. Where a burr hole is the means of accessing the brainstem, for example, stereotactic equipment suitable to aid in placement of an electrical stimulation lead on, in, or near the brainstem may be positioned around the head. Another alternative technique can include a modified midline or retrosigmoid posterior fossa technique.

In certain embodiments, electrical stimulation lead is located at least partially within or below the dura mater adjacent the brainstem. Alternatively, a stimulation lead can be placed in communication with the predetermined brainstem area by threading the stimulation lead up the spinal cord column, as described above, which is incorporated herein.

Still further, a predetermined brainstem area can be indirectly stimulated by implanting a stimulation lead in communication with a cranial nerve (e.g., olfactory nerve, optic, nerve, oculomotor nerve, trochlear nerve, trigeminal nerve, abducent nerve, facial nerve, vestibulocochlear nerve, glossopharyngeal nerve, vagal nerve, accessory nerve, and the hypoglossal nerve) as well as high cervical nerves (cervical nerves have anastomoses with lower cranial nerves) such that stimulation of a cranial nerve indirectly stimulates the predetermined brainstem tissue. Such techniques are further described in U.S. Pat. Nos. 6,721,603; 6,622,047; and 5,335,657, each of which is incorporated herein by reference.

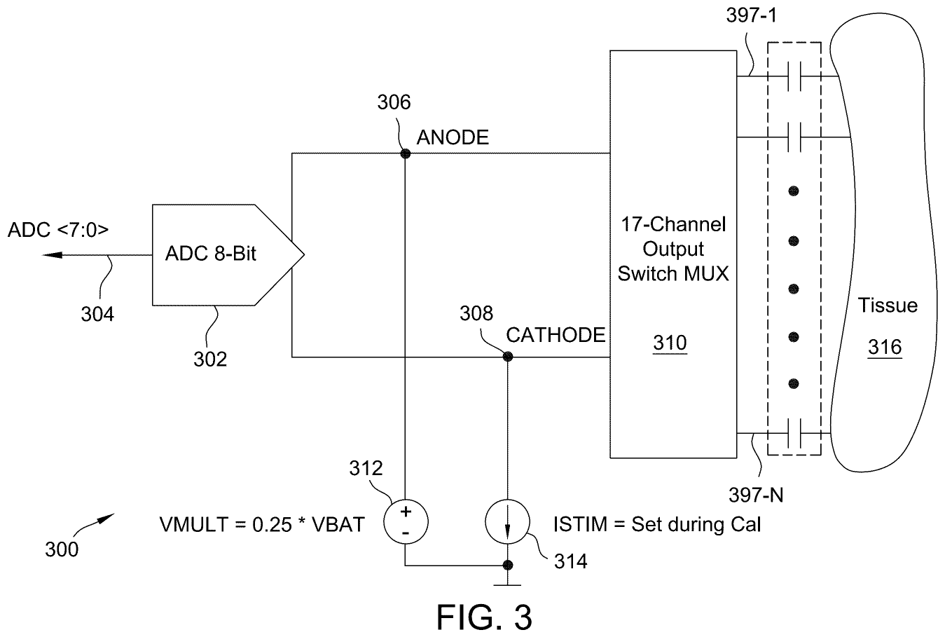

FIG. 3 depicts a diagnostic circuit portion 300 that may be configured to effectuate VRES measurements for purposes of some examples of the present disclosure. Skilled artisans will recognize that the diagnostic circuit portion 300 may be associated with or formed as a portion of an example IMD's pulse generator portion 100C described previously in reference to FIG. 1C. In an example implementation, an ADC block 302, e.g., an 8-bit ADC, without limitation as to any particular ADC resolution and/or dynamic range, may be coupled to a pair of electrical nodes configured as an anode 306 and a cathode 308 that may be energized in a cathodic stimulation of a patient's tissue 316. An output switching MUX 310 may be configured to couple the nodes 306, 308 to a pair of lead conductors selected from a plurality of lead conductors 397-1 to 397-N, which may be implanted near or onto the tissue 316. In one arrangement, a voltage multiplier 312 configured to provide adjustable control (e.g., generating a regulated voltage output in fractions of a battery voltage, VBAT) may be used for energizing the selected lead conductors during a stimulation pulse in order to effectuate a constant current (ISTIM) 314 for stimulation. In some implementations, ISTIM may be set during calibration of the IMD. Whereas the lead conductors 397-1 to 397-N may each be provided with a respective DC blocking capacitor in a baseline IMD implementation, they may be advantageously eliminated in an example embodiment of the present disclosure (as indicated by a dashed line box), wherein ADC 302 is configured to provide an output signal 304 to a diagnostic module (not shown in this FIG.) configured to effectuate VRES measurements, determine estimated VRES for the given stimulation settings, and provide appropriate diagnostics indications, and the like, in accordance with the teachings herein. Further, where the characterized Faradaic resistances (R_{F}) of the respective ETIs are determined to be nonnegligible, R_{F}-based threshold determinations may be implemented as part of a diagnostic monitoring scheme for detecting and indicating conditions in which excessive charge is being delivered in a stimulation scenario.

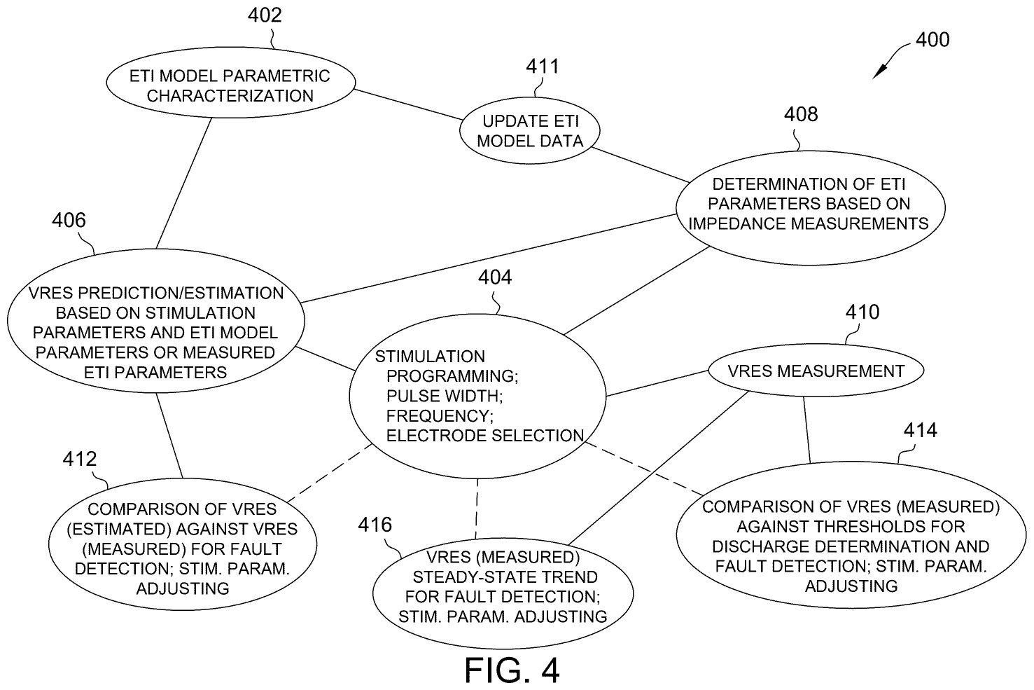

FIG. 4 depicts a VRES-based monitoring scheme for effectuating discharge mode selection and DC fault detection according to some examples of the present disclosure, wherein at least some portions may be executed asynchronously and/or at different locations either separately or in combination (e.g., on an IMD, at an external device, and/or at a network node deployed in a cloud-based digital healthcare infrastructure). In one arrangement, historical ETI load parametric characterization data may be obtained for a patient or a population of patients, which may be analyzed based on AI/ML techniques including pattern recognition, etc., as set forth at block 402. When a particular stimulation program is configured at the IMD for providing therapy (e.g., responsive to control signals from the patient, the clinician, or an authorized agent), applicable stimulation parameters such as pulse width, frequency, electrode selection, etc., may be selected (block 404). In one example arrangement, historical ETI load parameter data may be used in selecting appropriate resistive and reactive components (e.g., depending on ML-based pattern recognition) and using that data in conjunction with the stimulation settings data for determining estimated VRES. In another arrangement, the complex impedance measurement data obtained during the stimulation may be used in determining the resistive and reactive components, as set forth at block 408, which in turn may be used in determining estimated VRES for the given stimulation scenario. By way of illustration, various embodiments of obtaining estimated VRES values for given stimulation settings are set forth at block 406. In some embodiments, the historical ETI load parametric data may be updated, periodically or at configurable times, by the in situ complex impedance measurement data, as set forth at block 411. In some embodiments, the updated data may be used as training data and/or validation data in order to train and/or validate an AI/ML-based adaptive prediction engine configured to select appropriate ETI parametric values, which may be used in determining or estimating VRES for a given stimulation scenario. Further, measurements of VRES associated with the selected electrodes may be effectuated during the stimulation process in accordance with the embodiments set forth herein (block 410). One or more diagnostic monitoring and fault detection processes may be effectuated in response to the VRES measurements and estimated VRES predictions for the given stimulation settings, as indicated by blocks 412, 414, 416. Skilled artisans will recognize upon reference hereto that not all diagnostic monitoring and fault detection processes of the present disclosure need to be practiced, in any particular combination, sequence or order, for purposes of an example embodiment herein. Accordingly, various permutations, combinations or subcombinations of the processes described in the present disclosure may be implemented in an example arrangement. Further, an example diagnostic monitoring and fault detection process according to the teachings herein may be configured to generate appropriate feedback control signals to adjust one or more stimulation parameters (e.g., as exemplified by dashed line paths) and/or effectuate a corrective action such as, e.g., replacing an electrode, selecting a different lead, resetting the device, etc., as set forth elsewhere in the present disclosure. Still further, whereas the IMD device may be configured to perform the impedance measurements and VRES measurements in some example implementations, the determination of model parameters and calculation of what VRES should be (e.g., for a given stimulation setting) may be performed in the external device, e.g., the clinician programmer or patient controller.

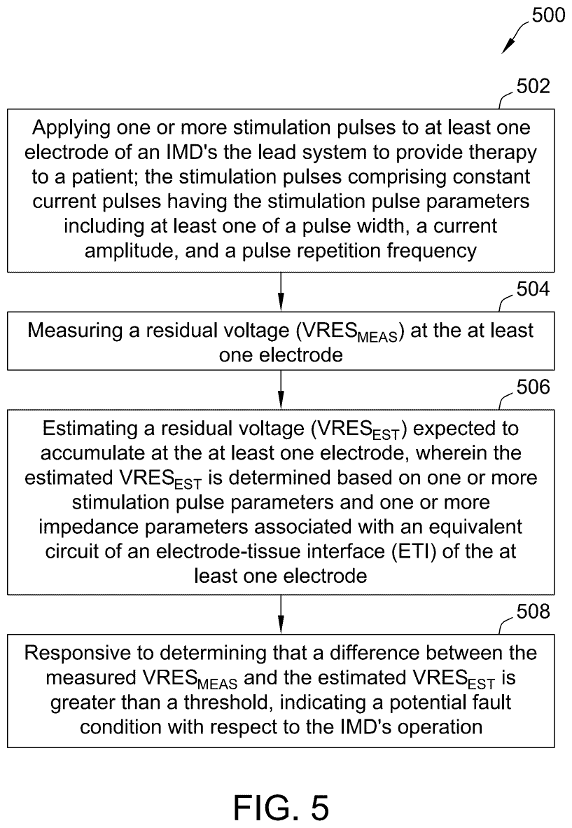

FIGS. 5-7 depict flowcharts illustrative of blocks, steps, functions and/or acts that may be (re)combined in one or more arrangements for effectuating a VRES-based diagnostic monitoring scheme according to some examples of the present disclosure. Reference numeral 500 of FIG. 5 generally refers to a method of operating an IPG/IMD device including one or more leads implanted proximate to a patient's tissue, wherein each lead includes a plurality of electrodes. The method may commence with selecting a set of stimulation parameters and applying one or more stimulation pulses to at least one electrode of the lead system according to the selected stimulation settings (block 502). At block 504, a residual voltage (VRES_{MEAS}) across the at least one electrode may be measured, e.g., on a pulse-by-pulse basis, relative to a reference node or another electrode depending on the programmed electrode configuration. At block 506, an estimated residual voltage (VRES_{EST}) expected to accumulate across the at least one electrode may be determined on an iterative basis over a plurality of pulses. As set forth in detail hereinabove, the estimated VRES_{EST} may be determined based on one or more stimulation pulse parameters and one or more impedance parameters associated with an equivalent ETI circuit representation of the at least one electrode. Responsive to determining that there exists a difference between the measured VRES_{MEAS} and the estimated VRES_{EST}, e.g., after a configurable number of pulses, and that the difference is greater than a threshold, a potential fault condition with respect to the IMD's operation may be indicated, which may relate to various aspects of the device hardware in an example embodiment as previously noted (block 508).

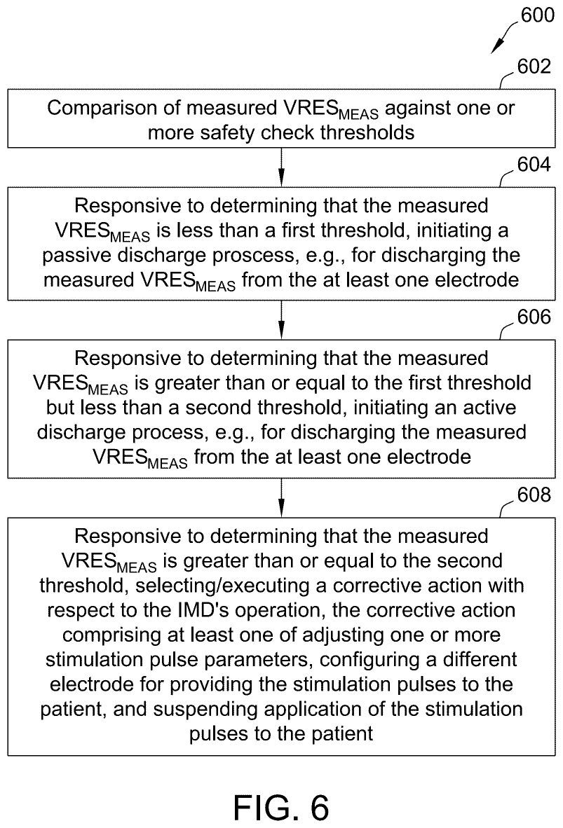

Another example method 600 of operating an IPG/IMD device is depicted in FIG. 6. As illustrated, method 600 may involve comparison of measured VRES_{MEAS} against one or more safety check thresholds, which in some examples may be configured as at least a portion of a decision-making process with respect to selecting a suitable discharge mode in a dynamic manner. Further, some examples may involve updating the safety check thresholds based on a learning process, which may be effectuated in association with other learning processes set forth in the present disclosure. Example process 600 may commence with obtaining suitable thresholds and comparing a measured VRES_{MEAS} against one or more safety check thresholds, which may be performed at a steady state, e.g., after applying a configurable number of pulses to the patient (block 602). Responsive to determining that the measured VRES_{MEAS} is less than a first configurable threshold, a passive discharge process may be initiated by the device, e.g., triggered by a control signal, for discharging the residual voltage from the at least one electrode (block 604). It should be appreciated that although there may be no DC blocking capacitors associated with the lead conductors in an example embodiment of the present disclosure, there is still capacitive charge buildup on the ETI, e.g., C_{DL}, which can be passively discharged in an example implementation.

In an additional and/or alternative arrangement, a further determination may be made with respect to a second configurable threshold in view of the measured VRES_{MEAS}. Responsive to determining that the measured VRES_{MEAS} is greater than or equal to the first threshold but less than the second threshold, a control signal may be generated for causing the discharge module of the device to initiate an active discharge process, e.g., for discharging the measured VRES_{MEAS} from the at least one electrode (block 606), which may be a balanced active discharge process or asymmetrical active discharge process. In a still further additional and/or alternative arrangement, responsive to determining that the measured VRES_{MEAS} is greater than or equal to the second threshold, a control signal may be generated for selecting/executing a corrective action with respect to the IMD's operation (block 608). As set forth previously, example corrective actions may comprise at least one of adjusting one or more stimulation pulse parameters, configuring a different electrode for providing the stimulation pulses to the patient, and suspending application of the stimulation pulses to the patient, wherein a prioritized sequence of corrective actions may be executed in some IMD device implementations.

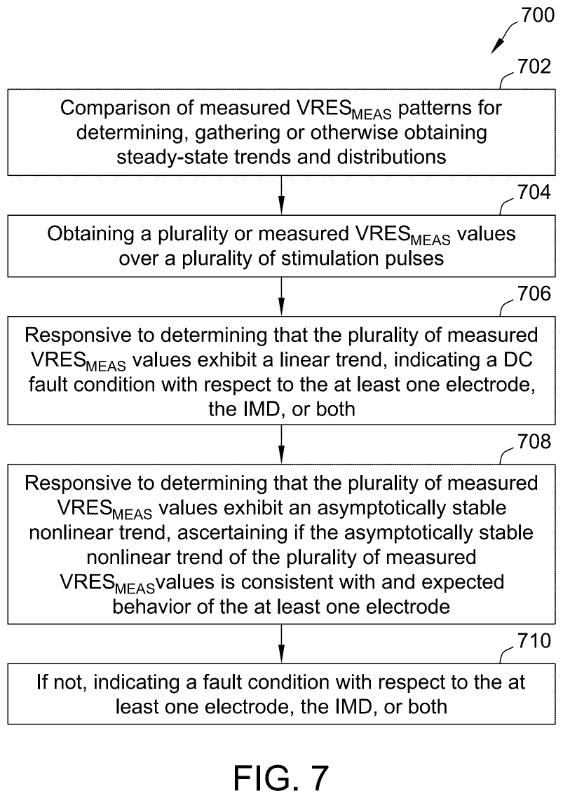

Another example method 700 of operating an IPG/IMD device is depicted in FIG. 7, wherein multiple measured VRES_{MEAS} values may be utilized for determining trends and distributions therein as set forth at block 702. As illustrated, method 700 may involve obtaining a plurality of measured VRES_{MEAS} values over a configurable number of stimulation pulses (block 704). Responsive to determining that the plurality of measured VRES_{MEAS} values exhibit a substantially linear trend, e.g., in an unbounded fashion, an indication, message or notification may be generated to indicate that a DC fault condition may exist with respect to at least one of the stimulated electrodes, the IMD device, or both (block 706). In an additional and/or alternative arrangement, a further determination may be made, e.g., after determining that the plurality of measured VRES_{MEAS} values do not exhibit a linear trend, as to whether the plurality of measured VRES_{MEAS} values exhibit an asymptotically stable nonlinear trend. If so, a verification may be generated ascertaining if the observed asymptotically stable nonlinear trend is consistent with the expected behavior of the stimulated electrodes (block 708). Otherwise, an indication, message or notification may be generated indicating fault condition with respect to at least one of the stimulated electrodes, the IPG/IMD device, or both (block 710).

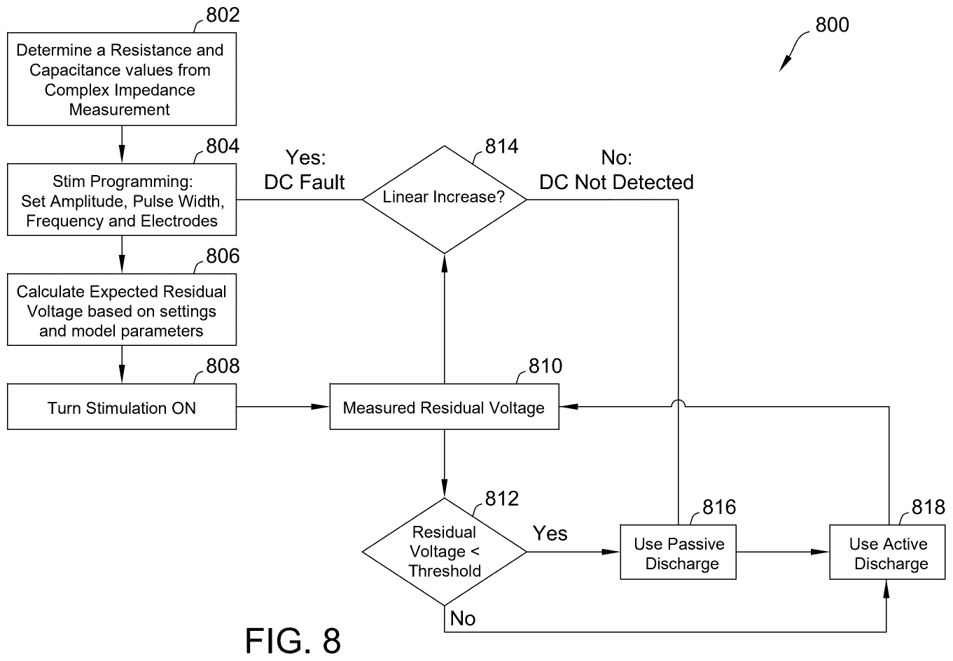

FIG. 8 depicts a flowchart illustrative of blocks, steps, functions and/or acts that may be (re)combined in one or more arrangements with other flowcharts according to some examples of the present disclosure. In one arrangement, example process 800 may be configured as a method of providing stimulation therapy to a patient. At block 802, ETI model parameters comprising resistance and capacitance values may be determined from complex impedance measurements obtained for the patient having an IMD device for therapy. At block 804, stimulation program parameters comprising amplitude, pulse width, frequency, etc., as well as electrode selection may be configured. At block 806, expected VRES may be calculated based on the configured stimulation settings and ETI model parameters. At block 808, stimulation to the patient may be turned on for providing therapy. At block 810, VRES may be measured with respect to the stimulated electrodes. Depending on the type of monitoring scheme implemented in a diagnostic scenario, one or more analyses concerning the device's operations may be effectuated. In one arrangement, a determination may be made as to whether a linear increase in the measured VRES is detected (block 814). If so, a DC fault may be indicated and a suitable corrective action, e.g., including stimulation adjustments, may be undertaken. Otherwise, a lack of DC fault condition may be indicated and a passive discharge scheme may be effectuated with respect to discharging the accumulated VRES from the stimulated electrodes, as set forth at block 816. In another arrangement, a discharge scheme selection may be made based on a configurable threshold. For example, if the measured VRES is greater than or equal to the threshold, an active discharge scheme may be employed, as set forth at blocks 812 and 818. Where an active discharge scheme is indicated, an example embodiment may be configured to determine the quantity of charge required (e.g., a minimum charge) to reduce the VRES to a pre-set value, e.g., 0 mV, 100 mV, etc. Otherwise, the passive discharge scheme may be employed in an example implementation (block 816). As noted previously, some of the foregoing acts may be performed asynchronously in some arrangements. Some additional and/or alternative embodiments of the present disclosure set forth further below describe a VRES-controlled active discharge scheme that may be configured to optionally include a passive discharge stage wherein a calculated/estimated minimum charge may be provided for discharging while minimizing or otherwise conserving battery power.

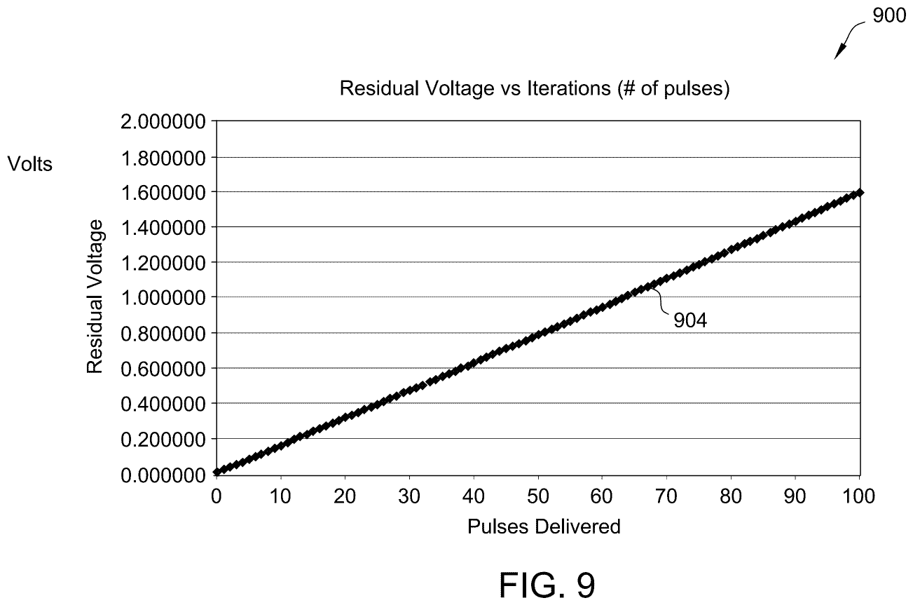

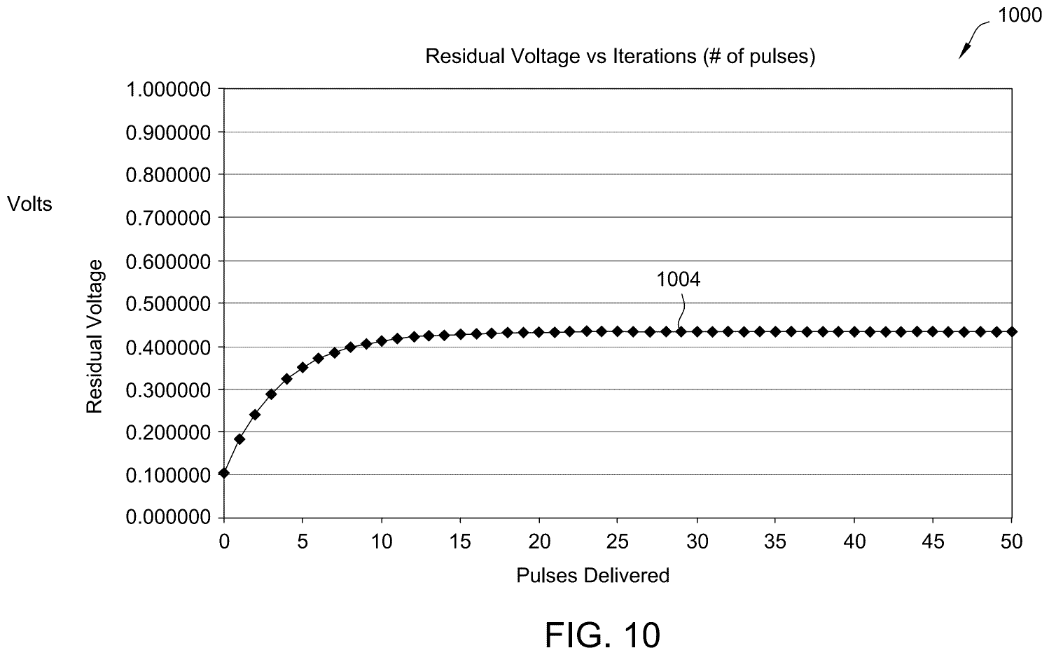

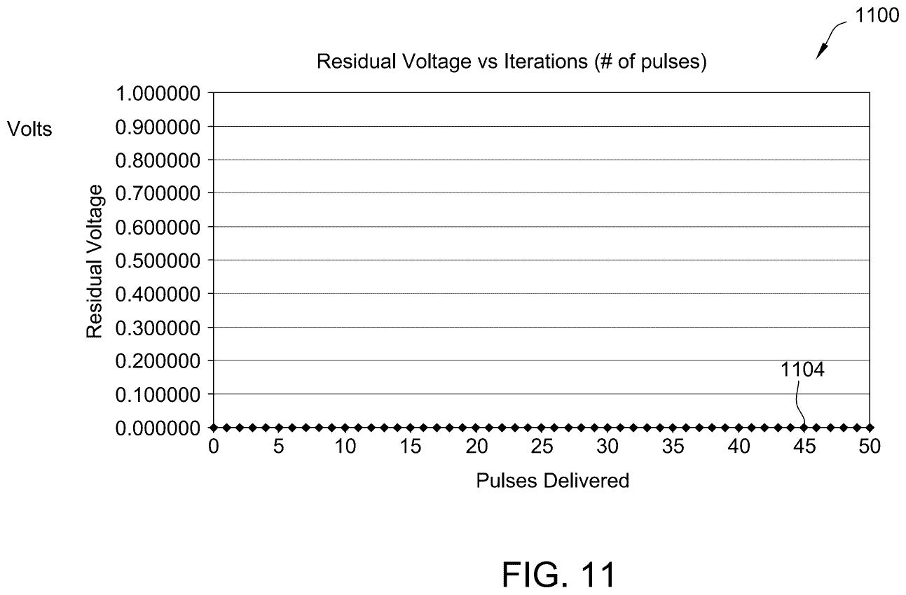

FIGS. 9-11 depict example VRES distributions or trends that may be used for diagnostic purposes according to some examples of the present disclosure. Graph 900 shown in FIG. 9 depicts a relationship between the number of stimulation pulses delivered and the measured VRES values in a therapy setting. A functional relationship 904 is illustrative of a linear increase, which may be used as an indicator for detecting DC fault conditions in an example IMD therapy scenario. Graph 1000 shown in FIG. 10 depicts an asymptotically stable nonlinear relationship 1004 between the number of delivered pulses and the measured VRES values, which may be used to detect a lack of DC fault condition in an example IMD therapy scenario. In some arrangements, the electrodes may be specifically designed for certain therapy applications, e.g., with particular form factors that have known or expected C_{DL} characteristics, that may be implanted in various tissue environments as noted previously in reference to FIGS. 2A-2J. Whereas it is expected that electrodes having larger C_{DL} values (e.g., SCS electrodes having C_{DL} values in the range of a few μF) exhibit asymptotically stable nonlinear trends in VRES measurements (similar to the relationship 1004 shown in FIG. 10), electrodes having smaller C_{DL} values (e.g., DBS electrodes having C_{DL} values in the range of several hundred nF) typically have little VRES accumulation and tend to exhibit substantially horizontal trends in steady-state conditions (i.e., with no slope), as illustrated by the relationship 1104 shown in graph 1100 of FIG. 11. Accordingly, an example embodiment of the present disclosure may be configured to provide a suitable diagnostic indication when an implanted electrode in a particular tissue environment exhibits a measured VRES behavior that is inconsistent with the expected behavior associated with the implanted electrode type in that environment.

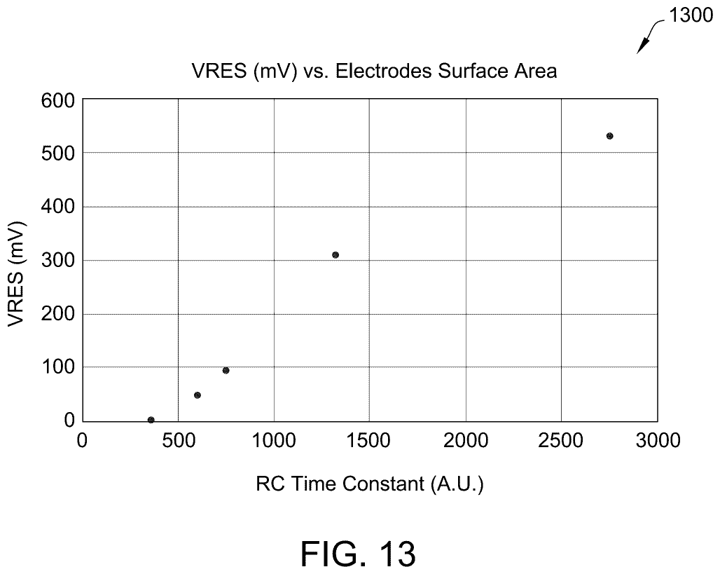

Further, an example embodiment of the present disclosure may be configured to utilize the relationship between VRES and the (effective) surface area of the electrodes in a stimulation setting scenario for diagnostic monitoring purposes, taken alone or in combination with one or more of the foregoing schemes. As the surface area of the electrode(s) decreases, VRES tends to decrease also. Because the ETI model parameters, R_{S} and C_{DL}, are proportional to the electrode surface area, a graphical relationship 1300 between an RC time constant (in arbitrary units or AUs, which can be equivalent to microseconds (μs) in some examples) and the calculated/predicted VRES (in mV), where all stimulation parameters held constant (as illustrated in FIG. 13), may be used to assess whether the implanted electrodes are operating as expected in a given environment.

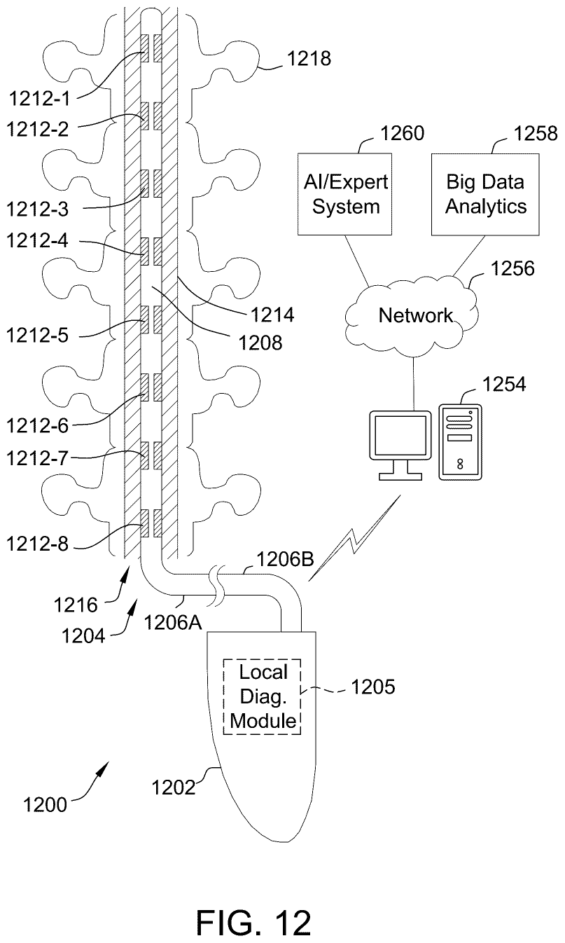

FIG. 12 illustrates a representative spinal cord stimulation (SCS) therapy application 1200 involving an IPG/IMD 1202 and associated lead system 1204 having a plurality of electrodes 1212-1 to 1212-8 wherein a VRES-based diagnostic monitoring scheme may be implemented according to an example embodiment of the present disclosure. In one arrangement, the lead system 1204 comprises a lead body 1206A/B coupled to an implantable lead 1208 that may be positioned at a desired target position in an epidural space 1216 defined by a plurality of vertebrae of a patient so as to be in close proximity to a nerve tissue of interest, e.g., a spinal cord 1214. The implantable lead 1208 includes eight electrodes 1212-1 to 1212-8, which may comprise ring electrodes, segmented or split electrodes, and the like, that may be separated from one another by equal or unequal portions of encapsulating material. The implantable lead 1208 is connected via lead body 1206A/1206B to the pulse generator or IMD 1202 that includes suitable diagnostic circuitry 1205 of the present disclosure configured to effectuate at least an embodiment of a VRES measurement and ETI characterization scheme set forth herein. At least a subset of the electrodes 1212-1 to 1212-8 may be selectively energized, i.e., stimulated to a target setting. In one arrangement, stimulation amplitudes and/or pulse widths may be incrementally ramped up in a ramping sequence (e.g., from low settings to the target values) to obtain the data required to calculate the resistive and capacitive components of the complex impedance in a manner set forth in U.S. Pat. No. 11,351,376, incorporated by reference hereinabove. In another arrangement, a sub-perception program may be used initially to obtain initial R and C values which can then be refined in further stimulation scenarios. In some arrangements, VRES measurements may be effectuated using current-carrying electrodes. Alternatively, VRES measurements may also be effectuated using suitable Kelvin connections as noted elsewhere in the present disclosure. Illustratively, electrodes 1212-1, 1212-4 and 1212-8 may be programmed as cathodes or anodes for operation in conjunction with the case or can of the IPG/IMD 1202 for providing current stimulation to effectuate an electric field that is spatially distributed over a target portion of the spinal cord 1214. An unused electrode, e.g., electrode 1212-5, may be used to establish a Kevin connection path on the inactive side of the measurement loop with respect to any of the selected active electrodes 1212-1, 1212-4 and 1212-8 for measuring induced and residual voltages associated therewith. Alternatively, any pair of current-carrying electrodes may be selectively coupled to the anodic and cathodic nodes of a measurement circuit (e.g., circuit 300 of FIG. 3) for effectuating VRES measurements as set forth above. Although a single implantable lead 1208 is exemplified herein, it should be appreciated that a lead system comprising multiple leads, each having a corresponding plurality of electrodes, may be implemented in a stimulation therapy application, wherein appropriate measurement data may be gathered for purposes of an example embodiment of the present disclosure.

In a further arrangement, the measured data may be transmitted via a suitable interface to an external node or device 1254 (e.g., a clinician programmer, a patient controller, etc.) that may be configured to execute processes relating to determining VRES estimates, ETI parametric extraction, diagnostic monitoring, etc., in order to reduce the computational load on the IMD/IPG 1202. In still further arrangements, external node 1254 may be configured as a communication gateway operative to provide the measured voltage data and/or ETI parametric data over a network 1256 to remote nodes such as expert systems 1260, Big Data analytics 1258, etc. to facilitate data mining, adaptive biostimulation therapy based on AI/ML etc., as set forth hereinabove.

In some additional and/or alternative embodiments, a VRES monitoring scheme effectuated by diagnostic circuitry such as, e.g., circuit 300, may be advantageously employed for optimizing discharge operations relative to an IPG/IMD device wherein a discharge pulse having only a minimum amount of charge necessary to reduce the measured VRES to a predetermined threshold, e.g., 0 mV, 100 mV, etc., may be generated as previously noted. It should be appreciated that in IPG/IMD therapy applications, delivery of charge-balanced stimulation pulses is typically required for ensuring long-term safety in tissue since net DC or unbounded charge buildup can lead to electrode corrosion and/or tissue damage. The current state of the art is to either use a passive discharge scheme, which allows for exponential decay based on the RC time constant of the implanted electrodes, or to use an active discharge program which delivers a charge equal to the therapy pulse charge in a reverse polarity. Whereas passive discharge is generally used at lower stimulation frequencies where there is sufficient time to remove voltage from the capacitors between pulses, an active discharge scheme may be configured to remove almost 100% of the built-up voltage in a charge-balanced pulsing arrangement. Typically, active discharge schemes may be implemented using high frequencies, which can increase power consumption by up to a factor of 2.

When an implanted system is configured to run an active discharge program, some embodiments may involve performing passive discharge operations also, e.g., during the time when the pulses aren't being delivered, which may be due to, e.g., potential mismatch in the delivery circuits, as well as due to leakage(s) that can occur in the implant system and/or at the electrode/tissue interface, etc. It should be appreciated that if there is some/sufficient time for passive discharge to completely or nearly 100% of the delivered charge, there may be no need for supplying active discharge pulses in some arrangements.

Some example embodiments of the present disclosure may therefore be configured to advantageously utilize a residual voltage measurement system in the implanted device in order to: (i) measure the steady-state residual voltage, i.e., VRES, existing prior to delivery of a next therapy pulse (or after a few therapy pulses, e.g., following a present therapy pulse), and (ii) adjust or modify the characteristics of an active discharge pulse to be applied (e.g., discharge pulse width and/or discharge pulse amplitude) in order to reduce the VRES to a pre-set value, e.g., VRES=0 mV, or some other clinically appropriate target value. Skilled artisans will recognize upon reference hereto that using the steady-state residual voltage for programming/adjusting the active discharge pulse parameters according to the examples herein saves battery current consumed in active discharge programs as it takes advantage of the passive discharge period(s) that may be interspersed during the program.

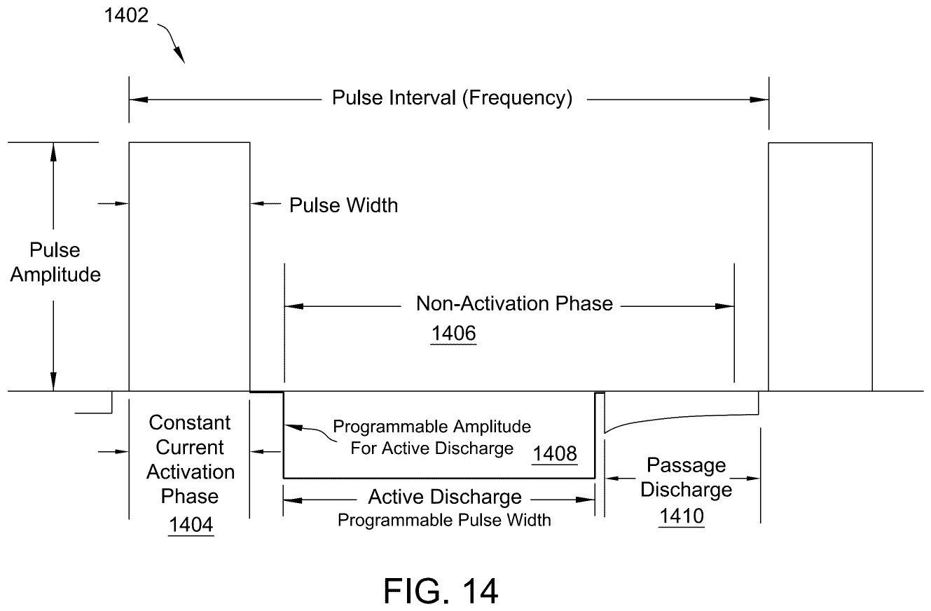

FIG. 14 depicts an example pulse train 1400 portion including a charge-balanced therapy pulse signal 1402 having an activation phase 1404 followed by a non-activation phase 1406, wherein an active discharge pulse 1408 having programmable/adjustable discharge pulse width and/or discharge pulse amplitude may be effectuated according to some embodiments of the present disclosure. A passive discharge stage 1410 may follow the active discharge pulse 1408 within the duration of the non-activation phase 1406 between two therapy pulses separated by a pulse interval. An example diagnostic circuit, e.g., circuit 300 described above in reference to FIG. 3, may be configured to monitor the residual voltage, wherein a controller, e.g., controller 112, 152 set forth above, may be configured to determine how much the charge to be delivered during the active discharge phase can be reduced, e.g., by adjusting either the discharge pulse width and/or the discharge pulse amplitude. In some implementations, an embodiment may therefore be configured to execute a closed-loop routine in the implanted device to reduce/modulate either or both of the discharge pulse parameters based on the VRES measurements.

In one arrangement, an example system that can use residual voltage as a means of controlling active discharge pulse parameters may be configured with appropriate circuitry and functionality, e.g., effectuated by or as controller 112/152 and circuitry associated therewith, to: (i) periodically measure the steady state residual voltage using an ADC or comparator based diagnostic circuit (e.g., circuit 300); and (ii) generate programmable amplitude and/or pulse width for the active discharge pulse, e.g., independent of the delivered therapy pulse(s), which may comprise cathodic pulses in some implementations as noted previously. In some arrangements, the charge delivered during the active discharge pulse (e.g., pulse 1408) in a non-activation phase 1406 can be less than the charge delivered during the activation phase 1404 due to the time spent in a passive discharge state (especially where the non-activation phase 1406 is long enough), which may be reduced by applying only a minimum amount of charge for discharging purposes. Example embodiments are therefore particularly advantageous in providing power savings due to delivering “just enough” charge during the active discharge pulse(s) of a charge-balanced pulse train of a therapy application. In cases where the stimulation frequency is low, power savings can be as much as a 50% reduction in total battery current according to some examples herein. Further, having the VRES measurement in the implanted device allows for the therapy system to adapt to changes in patient impedance over time, allowing an adaptive closed-loop system that may be configured to adjust the charge delivered during an active discharge phase automatically. It should be appreciated that significant power savings can be realized at higher frequencies as clinical data is gathered in response to allowing non-zero VRES results without negative impact to the tissue. Where the allowed VRES is increased (e.g., allowed VRES a predetermined non-zero threshold), additional (potentially greater) power savings may be realized in some implementations. It should be appreciated that pre-set values for VRES reduction may be determined based at least in part on a time period available for a passive discharge process during an inter-pulse interval in some example embodiments. Accordingly, higher pre-set VRES values may be configured where longer periods are available for passive discharge during non-activation phases of a charge-balanced pulse train according to the teachings herein. In some additional and/or alternative embodiments, a controller, e.g., controller 112/152, may therefore be advantageously configured to perform and/or facilitate, e.g., in association with diagnostic circuitry, a method involving a VRES-based modulated discharge cycling process, comprising, inter alia, applying one or more stimulation pulses to at least one electrode of the lead system; measuring a steady state residual voltage across the at least one electrode in reference to at least another electrode prior to delivery of a next stimulation pulse to the tissue; and modifying, responsive to the steady state residual voltage measurement, at least one of a discharge pulse amplitude and/or a discharge pulse width for generating a discharge pulse in an active discharge process operable to discharge the residual voltage to a pre-set value. In one arrangement, the example method may further comprise effectuating a passive discharge process following the active discharge process. In one arrangement, an active discharge process may be effectuated in response to a determination that the stead state residual voltage is greater than or equal to a threshold as noted previously. In a further arrangement, the example method may comprise determining a pre-set value to which the residual voltage may be reduced to that is based at least in part on a time period available for a passive discharge process during an inter-pulse interval.

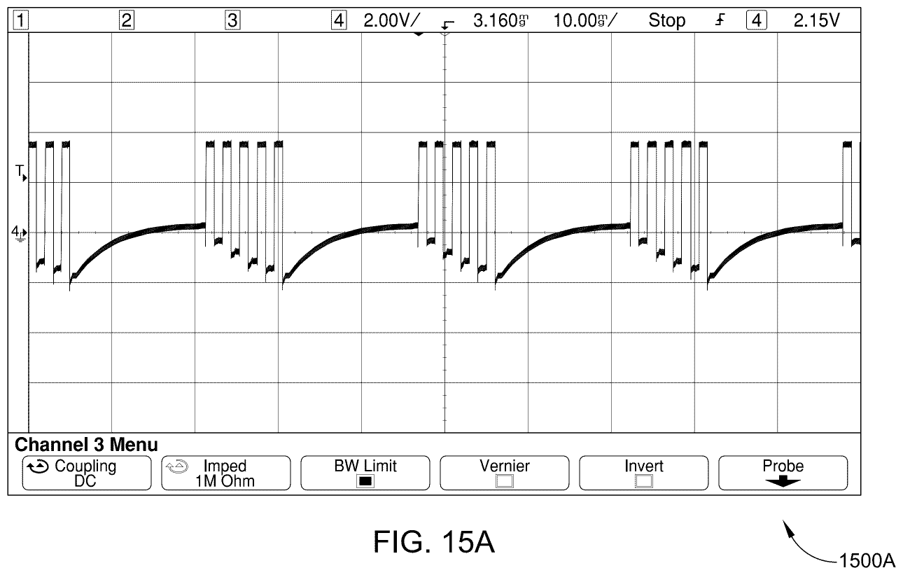

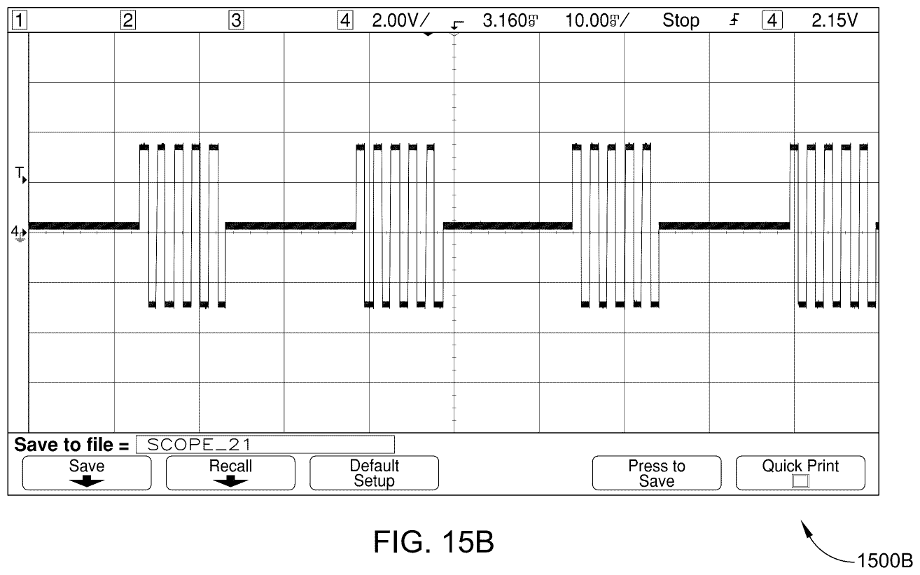

FIGS. 15A and 15B depict example pulse signal traces wherein a passive discharge scheme may be interspersed with an active discharge pulse consuming less battery power in some embodiments of an IPG/IMD according to the teachings herein. By way of illustration, reference numeral 1500A in FIG. 15A generally refers to a pulse train that includes a passive discharge phase between driven pulses, whereas reference numeral 1500B in FIG. 15B generally refers to a pulse train without a passive discharge phase. In both cases, a VRES of OV may be achieved although a lower charge for active discharge is consumed in the scenario shown in FIG. 15A.

Based on the foregoing Detailed Description, skilled artisans will recognize that embodiments of the present patent disclosure may be advantageously configured to utilize VRES measurements and predicted VRES determinations in a plethora of fault detection and discharge mode selection schemes that may be individualized on a patient by patient basis. Further, the disclosed schemes obviate the need for DC blocking capacitors, thereby allowing further miniaturization of IPG/IMD devices, which is advantageous in reducing or minimizing discomfort in patients during implant/explant procedures. Additional and/or cumulative benefits may therefore include, depending on implementation, one or several of the following without limitation: (i) a structure, apparatus or scheme to detect, in an IMD device, that delivered stimulation matches the programmed parameters; (ii) the capability to dynamically select the appropriate discharge mode (e.g., active vs. passive) based on the impedance of the individual ETI model for personalized therapy; (iii) the capability to deliver higher amplitudes in passive discharge to save battery current and extend the life of the device; (iv) a wider range of amplitude settings available in systems that are restricted to passive discharge for regulatory reasons; (v) the capability to detect DC leakage that could occur due to incorrect delivery of stimulation parameters, incorrect discharge and/or faults occurring at the implantable device outputs; and/or (vi) the ability to deliver a partial active discharge that just provides enough reverse current to sufficiently discharge electrodes, which can be useful for artifact reduction in a sensing system. In regard to the artifact reduction, it should be appreciated that because VRES monitoring could be used to set a minimum amount of charge for active discharge, battery current savings may be realized in an example implementation. Further, delivering “just enough” active discharge (instead of an amount equal to the forward pulse) may be configured to minimize the stimulation artifact(s) in a system attempting to sense biomarkers such as, e.g., evoked compound action potentials (ECAPs) and local field potentials (LFPs), which may serve as suitable closed-loop control signals in some stimulation applications.

In the above description of various embodiments of the present disclosure, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of this specification and the relevant art and may not be interpreted in an idealized or overly formal sense expressly so defined herein.

At least some example embodiments are described herein with reference to one or more circuit diagrams/schematics, block diagrams and/or flowchart illustrations. It is understood that such diagrams and/or flowchart illustrations, and combinations of blocks in the block diagrams and/or flowchart illustrations, can be implemented by any appropriate circuitry configured to achieve the desired functionalities. Accordingly, example embodiments of the present disclosure may be embodied in hardware and/or in software (including firmware, resident software, micro-code, etc.) operating in conjunction with suitable processing units or microcontrollers, which may collectively be referred to as “circuitry,” “a module” or variants thereof. An example processing unit or a module may include, by way of illustration, a general purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Field Programmable Gate Array (FPGA) circuits, any other type of integrated circuit (IC), and/or a state machine, as well as programmable system devices (PSDs) employing system-on-chip (SoC) architectures that combine memory functions with programmable logic on a chip that is designed to work with a standard microcontroller. Example memory modules or storage circuitry may include volatile and/or nonvolatile memories such as, e.g., random access memory (RAM), electrically erasable/programmable read-only memories (EEPROMs) or UV-EPROMS, one-time programmable (OTP) memories, Flash memories, static RAM (SRAM), etc.

Further, in at least some additional or alternative implementations, the functions/acts described in the blocks may occur out of the order shown in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Furthermore, although some of the diagrams include arrows on communication paths to show a primary direction of communication, it is to be understood that communication may occur in the opposite direction relative to the depicted arrows. Finally, other blocks may be added/inserted between the blocks that are illustrated.

It should therefore be clearly understood that the order or sequence of the acts, steps, functions, components or blocks illustrated in any of the flowcharts depicted in the drawing Figures of the present disclosure may be modified, altered, replaced, customized or otherwise rearranged within a particular flowchart, including deletion or omission of a particular act, step, function, component or block. Moreover, the acts, steps, functions, components or blocks illustrated in a particular flowchart may be inter-mixed or otherwise inter-arranged or rearranged with the acts, steps, functions, components or blocks illustrated in another flowchart in order to effectuate additional variations, modifications and configurations with respect to one or more processes for purposes of practicing the teachings of the present patent disclosure.

Although various embodiments have been shown and described in detail, the claims are not limited to any particular embodiment or example. None of the above Detailed Description should be read as implying that any particular component, element, step, act, or function is essential such that it must be included in the scope of the claims. Where the phrases such as “at least one of A and B” or phrases of similar import (e.g., “A and/or B”) are recited or described, such a phrase should be understood to mean “only A, only B, or both A and B.” Reference to an element in the singular is not intended to mean “one and only one” unless explicitly so stated, but rather “one or more.” Moreover, the terms “first,” “second,” and “third,” etc. employed in reference to elements or features are used merely as labels, and are not intended to impose numerical requirements, sequential ordering or relative degree of significance or importance on their objects. All structural and functional equivalents to the elements of the above-described embodiments that are known to those of ordinary skill in the art are expressly incorporated herein by reference and are intended to be encompassed by the present claims. Accordingly, those skilled in the art will recognize that the exemplary embodiments described herein can be practiced with various modifications and alterations within the spirit and scope of the claims appended below.

1. An implantable pulse generator (IPG) for generating electrical pulses for application to tissue of a patient, comprising:
a controller for controlling operations of the IPG;
a battery for powering the IPG;
circuitry for generating electrical pulses;
a header for connecting to one or more stimulation leads with a plurality of electrodes; and
diagnostic circuitry coupled to the controller and the circuitry for generating electrical pulses, the diagnostic circuitry configured to measure a residual voltage (VRES_{MEAS}) across at least one electrode in reference to at least another electrode,
wherein the controller is configured to estimate a residual voltage (VRES_{EST}) expected to accumulate across the at least one electrode, wherein the estimated VRES_{EST} is determined based on one or more stimulation pulse parameters and one or more impedance parameters associated with an equivalent circuit of an electrode-tissue interface (ETI) of the at least one electrode, and wherein the controller is configured to, responsive to determining that a difference between the measured VRES_{MEAS} and the estimated VRES_{EST} is greater than a threshold, indicate a potential fault condition with respect to the IPG's operation.

2. The IPG as recited in claim 1, wherein the potential fault condition is indicative of a fault with the at least one electrode, the IPG, or both.

3. The IPG as recited in claim 1, wherein each electrode of the plurality of electrodes is coupled to a respective conductive trace disposed in a stimulation lead without a DC locking capacitor (C_{DC}).

4. The IPG as recited in claim 1, wherein the electrical pulses comprise constant current pulses having stimulation pulse parameters including at least one of a pulse width, a current amplitude, and a pulse repetition frequency.

5. The IPG as recited in claim 1, wherein the controller is further configured to:
perform, responsive to determining that the measured VRES_{MEAS} is less than a first threshold, initiating a passive discharge process for discharging the measured VRES_{MEAS} from the at least one electrode;
perform, responsive to determining that the measured VRES_{MEAS} is greater than or equal to the first threshold but less than a second threshold, initiating an active discharge process for discharging the measured VRES_{MEAS} from the at least one electrode; and
determine a minimum quantity of charge required for the active discharge process to reduce the measured VRES_{MEAS} to a pre-set value.

6. (canceled)

7. (canceled)

8. The IPG as recited in claim 5, wherein the controller is further configured to perform, responsive to determining that the measured VRES_{MEAS} is greater than or equal to the second threshold, selecting a corrective action with respect to the IPG's operation, the corrective action comprising at least one of adjusting one or more stimulation pulse parameters, configuring a different electrode for providing the stimulation pulses to the patient, and suspending application of the stimulation pulses to the patient.

9. The IPG as recited in claim 1, wherein the controller is further configured to perform:
obtaining a plurality of measured VRES_{MEAS} values over a plurality of stimulation pulses;
responsive to determining that the plurality of measured VRES_{MEAS} values exhibit a linear trend, indicating a DC fault condition with respect to the at least one electrode, the IPG, or both.

10. The IPG as recited in claim 1, wherein the controller is further configured to perform:
obtaining a plurality of measured VRES_{MEAS} values over a plurality of stimulation pulses;
responsive to determining that the plurality of measured VRES_{MEAS} values exhibit an asymptotically stable nonlinear trend, ascertaining if the asymptotically stable nonlinear trend of the plurality of measured VRES_{MEAS} values is consistent with an expected behavior of the at least one electrode; and
otherwise, indicating a fault condition with respect to the at least one electrode, the IPG, or both,
wherein the electrical pulses comprise at least one of monophasic stimulation pulses, biphasic stimulation pulses, burst stimulation pulses and tonic stimulation pulses.

11. (canceled)

12. A method of operating an implantable pulse generator (IPG) including one or more leads implanted proximate to a patient's tissue, wherein each lead includes a plurality of electrodes, the method comprising:
applying one or more stimulation pulses to at least one electrode of the plurality of electrodes;
measuring a residual voltage (VRES_{MEAS}) across the at least one electrode in reference to at least another electrode;
estimating a residual voltage (VRES_{EST}) expected to accumulate across the at least one electrode, wherein the estimated VRES_{EST} is determined based on one or more stimulation pulse parameters and one or more impedance parameters associated with an equivalent circuit of an electrode-tissue interface (ETI) of the at least one electrode; and
responsive to determining that a difference between the measured VRES_{MEAS} and the estimated VRES_{EST} is greater than a threshold, indicating a potential fault condition with respect to the IPG's operation.

13. The method as recited in claim 12, wherein the potential fault condition is indicative of a fault with the at least one electrode, the IPG, or both.

14. The method as recited in claim 12, wherein each electrode of the plurality of electrodes is coupled to a respective conductive trace disposed in the lead without a DC blocking capacitor (C_{DC}).

15. The method as recited in claim 12, wherein the one or more stimulation pulses comprise constant current pulses having stimulation pulse parameters including at least one of a pulse width, a current amplitude, and a pulse repetition frequency.

16. The method as recited in claim 12, further comprising:
responsive to determining that the measured VRES_{MEAS} is less than a first threshold, initiating a passive discharge process for discharging the measured VRES_{MEAS} from the at least one electrode;
responsive to determining that the measured VRES_{MEAS} is greater than or equal to the first threshold but less than a second threshold, initiating an active discharge process for discharging the measured VRES_{MEAS} from the at least one electrode; and
determining a minimum quantity of charge required for the active discharge process to reduce the measured VRES_{MEAS} to a pre-set value.

17. (canceled)

18. (canceled)

19. The method as recited in claim 16, further comprising, responsive to determining that the measured VRES_{MEAS} is greater than or equal to the second threshold, selecting a corrective action with respect to the IPG's operation, the corrective action comprising at least one of adjusting one or more stimulation pulse parameters, configuring a different electrode for providing the stimulation pulses to the patient, and suspending application of the stimulation pulses to the patient.

20. The method as recited in claim 12, further comprising:
obtaining a plurality of measured VRES_{MEAS} values over a plurality of stimulation pulses; and
responsive to determining that the plurality of measured VRES_{MEAS} values exhibit a linear trend, indicating a DC fault condition with respect to the at least one electrode, the IPG, or both.

21. The method as recited in claim 12, further comprising:
obtaining a plurality of measured VRES_{MEAS} values over a plurality of stimulation pulses;
responsive to determining that the plurality of measured VRES_{MEAS} values exhibit an asymptotically stable nonlinear trend, ascertaining if the asymptotically stable nonlinear trend of the plurality of measured VRES_{MEAS} values is consistent with an expected behavior of the at least one electrode; and
otherwise, indicating a fault condition with respect to the at least one electrode, the IPG, or both,
wherein the stimulation pulses comprise at least one of monophasic pulses, biphasic stimulation pulses, burst stimulation pulses and tonic stimulation pulses.

22. (canceled)

23. An implantable pulse generator (IPG) for generating electrical pulses for application to tissue of a patient, comprising:
a controller for controlling operations of the IPG;
a battery for powering the IPG;
circuitry for generating electrical pulses;
a header for connecting to one or more stimulation leads with a plurality of electrodes; and
diagnostic circuitry coupled to the controller and the circuitry for generating electrical pulses, the diagnostic circuitry configured to measure a steady state residual voltage across at least one electrode in reference to at least another electrode prior to delivery of a next electrical pulse to the tissue,
wherein the controller is configured to modify, responsive to the steady state residual voltage measurement, at least one of a discharge pulse amplitude and a discharge pulse width for generating a discharge pulse in an active discharge process operable to discharge the residual voltage to a pre-set value.

24. The IPG as recited in claim 23, wherein the controller is configured to effectuate a passive discharge process following the active discharge process.

25. The IPG as recited in claim 23, wherein the controller is configured to effectuate the active discharge process in response to a determination that the steady state residual voltage is greater than or equal to a threshold.

26. (canceled)

27. (canceled)

28. The IPG as recited in claim 23, wherein the pre-set value is determined based at least in part on a time period available for a passive discharge process during an inter-pulse interval.

29-32. (canceled)
